(19) United States
(12) Patent Application Publication
(10) Pub. No.: US 2012/0040904 A1
(43) Pub. Date: Feb. 16, 2012

(54) Boronate Medicaments for Preventing Thrombosis During Surgery

(75) Inventors: Sophie Marie Combe-Marzelle, London (GB); Sanjay Kumar Kakkar, London (GB); Graham Douglas Allen, Ancaster (GB)

(73) Assignee: Trigen Holdings AG, London (GB)

(21) Appl. No.: 10/592,265

(22) PCT Filed: Mar. 9, 2005
(86) PCT No.: PCT/GB2005/000908

§ 371 Date: Sep. 21, 2007

(30) Foreign Application Priority Data
Mar. 9, 2004 (GB) 0405280.9

(51) Int. Cl. A61K 38/00; A61K 31/69; A61K 38/36

(52) U.S. Cl. 514/13.7; 514/64

(57) ABSTRACT

The use for the manufacture of a medicament for preventing unwanted coagulation during surgery, and particularly a Coronary Artery By pass Graft (CABG) procedure, of boronic acids of formula (I), and salts, prodrugs and prodrug salts thereof; wherein Y comprises a moiety which, together with the fragment —CH(R⁹)—B(OH)₂, has affinity for the substrate binding site of thrombin; and R⁹ is a straight chain alkyl group interrupted by one or more ether linkages and in which the total number of oxygen and carbon atoms is (3, 4, 5) or (6) or R⁹ is —(CH₂)ₘ—W where m is (2, 3, 4) or (5) and W is —OH or halogen (F, CI, Br or I).

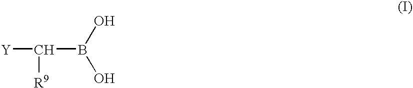

### BACKGROUND

The present disclosure relates to pharmaceutically useful products obtainable from organoboronic acids. The disclosure also relates to the use of members of the aforesaid class of products, in particular in relation to surgery and the like.

The disclosure further relates to parenteral pharmaceutical formulations containing the described products, and to other subject matter.

### Boronic Acid Compounds

It has been known for some years that boronic acid compounds and their derivatives, e.g. esters, have biological activities, notably as inhibitors or substrates of proteases. 2. In describing inhibitors or substrates of proteases, P1, P2, P3, etc. designate substrate or inhibitor residues which are amino-terminal to the scissile peptide bond, and S1, S2, S3, etc., designate the corresponding subsites of the cognate protease in accordance with: Schechter, I. and Berger, A. On the Size of the Active Site in Proteases, Biochem. Biophys. Res. Comm., 27:157-162, 1967. In thrombin, the S1 binding site or “specificity pocket” is a well defined groove in the enzyme, whilst the S2 and S3 binding subsites (also respectively called the proximal and distal hydrophobic pockets) are hydrophobic and interact strongly with, respectively, Pro and (R)-Phe, amongst others.

Pharmaceutical research into serine protease inhibitors has moved from simple arylboronic acids to boropeptides, i.e. peptides containing a boronic acid analogue of an α-amino carboxylic acid. The boronic acid may be derivatised, often to form an ester. Shenvi (EP-A-145441 and U.S. Pat. No. 4,499,082) disclosed that peptides containing an α-aminoboronic acid with a neutral side chain were effective inhibitors of elastase and has been followed by numerous patent publications relating to boropeptide inhibitors of serine proteases.

Aminoboronate or peptidoboronate inhibitors or substrates of serine proteases are described in:
U.S. Pat. No. 4,935,493
EP 341661
WO 94/25049
WO 95/09859
WO 96/12499
WO 96/20689
Lee S-L et al, Biochemistry 36:13180-13186, 1997
Dominguez C et al, Bioorg. Meld. Chem. Lett. 7:79-84, 1997
EP 471651
WO 94/20526
WO 95/20603
WO97/05161
U.S. Pat. No. 4,450,105
U.S. Pat. No. 5,106,948
U.S. Pat. No. 5,169,841
WO 96/25427
U.S. Pat. No. 5,288,707
WO 96/20698
WO 01/02424.

The amino acid sequence (R)-Phe-Pro-Arg, imitating amino acid sequences of fibrinogen, was at one time considered the best sequence for thrombin inhibitors. This sequence formed tight-binding inhibitors of thrombin, e.g. Ac-(R)-Phe-Pro-boroArg (DUP 714), having Ki values in the picomolar range (Kettner et al, J. Biol. Chem. 265: 18289-18297, 1990; EP-A-293,881).

The replacement of the P2 Pro residue of borotripeptide thrombin inhibitors by an N-substituted glycine is described in Fevig J M et al Bioorg. Med. Chem. 8: 301-306 and Rupin A et al Thromb. Haemost 78(4):1221-1227, 1997. See also U.S. Pat. No. 5,585,360 (de Nanteuil et al).

Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Unfortunately, organoboronic acids can be relatively difficult to obtain in analytically pure form. Thus, alkylboronic acids and their boroxines are often air-sensitive. Korcek et al, J. Chem. Soc. Perkin Trans. 2:242, 1972, teaches that butylboronic acid is readily oxidized by air to generate 1-butanol and boric acid.

It is known that derivatisation of boronic acids as cyclic esters provides oxidation resistance. For example, Martichonok V et al J. Am. Chem. Soc. 118: 950-958, 1996 state that diethanolamine derivatisation provides protection against possible boronic acid oxidation. U.S. Pat. No. 5,681,978 (Matteson D S et al) teaches that 1,2-diols and 1,3 diols, for example pinacol, form stable cyclic boronic esters that are not easily oxidised.

WO 02/059131 discloses boronic acid products which are described as stable. In particular, these products are certain boropeptides and/or boropeptidomimetics in which the boronic acid group has been derivatised with a sugar, e.g. mannitol, to form a sugar ester.

### Thrombosis

Hemostasis is the normal physiological condition of blood in which its components exist in dynamic equilibrium. When the equilibrium is disturbed, for instance following injury to a blood vessel, certain biochemical pathways are triggered leading, in this example, to arrest of bleeding via clot formation (coagulation). Coagulation is a dynamic and complex process in which proteolytic enzymes such as thrombin play a key role. Blood coagulation may occur through either of two cascades of zymogen activations, the extrinsic and intrinsic pathways of the coagulation cascade. The last protease in each pathway is thrombin, which catalyses the polymerization of fibrinogen monomers to fibrin polymer.

The management of thrombosis commonly involves the use of antiplatelet drugs (inhibitors of platelet aggregation) to control future thrombogenesis and thrombolytic agents to lyse the newly formed clot, either or both such agents being used in conjunction or combination with anticoagulants. Anticoagulants are used also preventatively (prophylactically) in the treatment of patients thought susceptible to thrombosis.

Currently, two of the most effective classes of drugs in clinical use as anticoagulants are the heparins and the vitamin K antagonists. The heparins are ill-defined mixtures of sulfated polysaccharides that bind to, and thus potentiate, the action of antithrombin III. Antithrombin III is a naturally occurring inhibitor of the activated clotting factors IXa, Xa, XIa, thrombin and probably XIIa (see Jaques, Pharmacol. Rev. 31:99-166, 1980). The vitamin K antagonists, of which warfarin is the most well-known example, act indirectly by inhibiting the post-ribosomal carboxylations of the vitamin K dependent coagulation factors II, VII, IX and X (see Hirsch, Semin. Thromb. Hemostasis 12:1-11, 1986). While effective therapies for the treatment of thrombosis, heparins and vitamin K antagonists have the unfortunate side effects of bleeding, heparin-induced thrombocytopenia (in the case of heparin) and marked interpatient variability, resulting in a small and unpredictable therapeutic safety margin.

It is therefore desirable to have parenteral direct thrombin inhibitors without the problems associated with heparin.

Many organoboronic acid compounds may be classified as lipophilic or hydrophobic. Typically, such compounds include amongst others:
boropeptides of which all or a majority of the amino acids are hydrophobic
boropeptides of which at least half of the amino acids are hydrophobic and which have a hydrophobic N-terminal substituent (amino protecting group)
non-peptides based on hydrophobic moieties.

The use of direct acting inhibitors of thrombin and other serine protease enzymes of the coagulation system is expected to alleviate these problems. To that end, a wide variety of serine protease inhibitors have been tested, including boropeptides, i.e. peptides containing a boronic acid analogue of an α-amino acid. Whilst direct acting boronic acid thrombin inhibitors have been discussed earlier in this specification, they are further described in the following section.

### Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TR150b). The corresponding free boronic acid is known as TRI 50c. For further information relating to TR150b and related compounds, the reader is referred to the following documents:
Elgendy S et al., in The Design of Synthetic Inhibitors of Thrombin, Claeson G et al Eds, Advances in Experimental Medicine, 340:173-178, 1993.
Claeson G et al, Biochem J. 290:309-312, 1993
Tapparelli C et al, J Biol Chem, 268:4734-4741, 1993
Claeson G. in The Design of Synthetic Inhibitors of Thrombin, Claeson G et al Eds, Advances in Experimental Medicine, 340:83-91, 1993
Phillip et al, in The Design of Synthetic Inhibitors of Thrombin, Claeson G et al Eds, Advances in Experimental Medicine, 340:67-77, 1993
Tapparelli C et al, Trends Pharmacol. Sci. 14:366-376, 1993
Claeson G, Blood Coagulation and Fibrinolysis 5:411-436, 1994
Elgendy et al, Tetrahedron 50:3803-3812, 1994
Deadman I et al, J. Enzyme Inhibition 9:29-41, 1995
Deadman J et al, J. Medicinal Chemistry 38:1511-1522, 1995.

TRI 50b is considered to be a prodrug for TRI 50c, which is the active principal in vivo. The tripeptide sequence of TRI 50c has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. Thus, the active, or most active, TRI 50c stereoisomer is considered to be of R,S,R configuration and may be represented as:

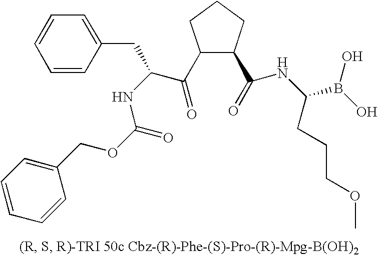

WO 2004/022072, and also U.S. Ser. No. 10/659,178 and EP-A-1396270, disclose pharmaceutically acceptable base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a pharmaceutically acceptable base addition salt of a boronic acid of, for example, formula (A):

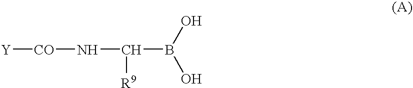

wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms. Salts of TRI 50c are exemplary.

WO 2004/022071, and also U.S. Ser. No. 10/659,179 and EP-A-1396269, disclose salts of a pharmaceutically acceptable multivalent (at least divalent) metal and an organoboronic acid drug. Such salts are described as having an improved level of stability which cannot be explained or predicted on the basis of known chemistry, and as being indicated to have unexpectedly high and consistent oral bioavailability not susceptible of explanation on the basis of known mechanisms. The oral formulations of such salts are therefore also disclosed.

One particular class of salts comprises those wherein the organoboronic acid comprises a boropeptide or boropeptidomimetic. Such drugs which may beneficially be prepared as salts include without limitation those of the formula X-(aa)ₙ-B(OH)₂, where X is H or an amino-protecting group, n is 2, 3 or 4, (especially 2 or 3) and each aa is independently a hydrophobic amino acid, whether natural or unnatural. In one class of multivalent metal salts, the organoboronic acid is of formula (A) above. Salts of TRI 50c are exemplary.

WO 2004/022070, and also U.S. Ser. No. 10/658,971 and EP-A-1400245, disclose and claim inter alia parenteral pharmaceutical formulations that include a pharmaceutically acceptable base addition salt of a boronic acid of, for example, formula (A) above. Such salts are described as having an improved level of stability which cannot be explained or predicted on the basis of known chemistry. Salts of TRI 50c are exemplary.

### Rapid Onset Anticoagulation and Rapid Offset Anticoagulation

It is desirable in some circumstances to administer an anticoagulant whose onset and offset of activity can be closely controlled. Thus, some anticoagulants will have an excessively long duration of activity after administration for a particular indication; one example is that warfarin takes a long time to reduce from therapeutic levels of activity. Whereas this may be desired for some therapeutic uses, it is far from ideal in e.g. some surgical procedures, where anticoagulation is desired during surgery but effectively normal coagulant activity is desired as soon as surgery is finished. Long duration drugs can cause severe post-operational difficulties including bleeding and are therefore not suited to uses in procedures where therapeutic levels may need to be adjusted or controlled.

It is envisaged that a compound having a rapid offset, or quick “switch-off”, will need to be continually administered, for example by infusion, while a therapeutic level of activity is required. The level of therapeutic activity will be controllable by adjusting the dose or rate of infusion. It is envisaged that a compound having rapid offset will enable the therapeutic level to be reduced with relatively fast effect by way of reducing the dose or flow rate the patient receives. The dose could be controlled in relation to the prevailing circumstances.

It is also envisaged that when the therapeutic level of activity is no longer required, e.g. the operation has been completed, or there are complications such as adverse reactions to the anticoagulant, the therapeutic level will decrease rapidly once administration has ceased.

In the case of emergency anticoagulant administration, it will often be desired as a short-term precautionary measure to administer an anticoagulant to a patient who has suffered, or is suspected of having suffered, a thrombotic event. In such an emergency, the anticoagulant could be administered prior to admission to hospital or immediately upon admission, in order to protect the patient against a further possible thrombotic event before a detailed examination of the patient can be undertaken. A short duration of activity is highly desirable in case it subsequently proves undesirable for the patient to be anticoagulated, as for example where a course of treatment is to be followed (e.g. surgery) where presence of a significant amount of anticoagulant in the blood could be dangerous or where medication is given which is incompatible with the emergency anticoagulant.

Anticoagulation agents are particularly required for procedures involving an extracorporeal blood circuit, for example Coronary Artery Bypass Graft (CABG) surgery. According to the American Heart Association, currently more than 500,000 CABG procedures are performed annually in the US, and more than 700,000 are performed worldwide. The present anticoagulant used in all CABG procedures is heparin.

Heparin has significant limitations and can cause, for example, bleeding and heparin-induced thrombocytopenia (HIT) and is difficult to dose accurately and requires dose monitoring. Heparin does not have a short half life in the body and will cause post-operative bleeding. Consequently a CABG patient must receive not only heparin during the operation but also a reversal agent subsequently, to bring the patient's coagulation levels back to a safe, non-therapeutic level. The reversal agent for heparin, protamine, carries the risk of significant side effects.

Protamine is the only drug available to reverse heparin but has never been approved by the FDA. Heparin has been associated with systemic hypotension as well as pulmonary hypertension, both of which can lead to life threatening complications. Protamine has also been associated with platelet dysfunction, complement activation and thrombus formation, which effects can cause excessive bleeding, organ dysfunction and stroke respectively.

In published medical journals, protamine has been associated with a variety of adverse events that can lead to costly and potentially life threatening complications:
Stephen E Kimmel et al, Mortality and adverse events after protamine administration in patients undergoing cardiopulmonary bypass, Cardiovascular Anaesthesia, 2002, 94, 402-8
J A Carr et al, The heparin protamine interaction, The Journal of Cardiovascular Surgery, 1999, 40, 659-66
Stephen E Kimmel et al, Adverse events after protamine administration in patients undergoing cardiopulmonary bypass: risks and predictors of underreporting, Journal of Clinical Epidemiology, 1998, 51, 1-10
Michael C Mauney et al, Stroke rate is markedly reduced after carotid endarterectomy by avoidance of protamine, Journal of Vascular Surgery, 1995, 22, 264-270.

As a result of the difficulties surrounding heparin, Low Molecular Weight Heparins (LMWHs) such as lovenox and pentasaccharides such as arixtra are now being used to manage a variety of thrombotic, cardiovascular, orthopaedic surgery and metastatic disorders. However, protamine is less effective as a reversal agent for the LMWHs and is not effective on synthetic heparin-like pentasaccharides. There therefore remains a need for an anticoagulant which can be used to prevent thrombosis or unwanted coagulation during surgery and which does not require a reversal agent having the side effects of protamine.

### BRIEF SUMMARY OF THE DISCLOSURE

The disclosure relates to boronic acids and their derivatives, and to the use of such compounds. There are provided novel methods, uses and compounds.

The disclosure relates, in one aspect, to a method of inhibiting thrombin comprising administering parenterally to a subject in need thereof a therapeutically effective amount of a boronic acid compound disclosed herein, e.g. a pharmaceutically acceptable base addition salt of a boronic acid which has a neutral thrombin P1 domain linked to a hydrophobic moiety capable of binding to the thrombin S2 and S3 subsites. The boronic acid compounds may be, for example, in the form of the free acid, an acid addition salt, a base addition salt or a prodrug.

Embodiments of the disclosure relate to prevention of thrombosis or unwanted coagulation during surgical operations, for example in procedures involving an extracorporeal blood circuit. An example of an extracorporeal blood circuit is a cardiopulmonary bypass circuit.

An example of a surgical procedure relating to the disclosure is a coronary artery bypass graft (CABG) procedure. Coronary artery bypass grafting is a procedure to bypass obstructions of the coronary arteries. During CABG, the patient may be connected to a cardiopulmonary bypass machine so that the heart can be stopped during surgery, or a cardiopulmonary bypass machine may be dispensed with. The invention relates amongst other things to CABG procedures both with and without the use of a cardiopulmonary bypass machine. The invention therefore relates to formulations containing the disclosed compounds for use in preventing coagulation during a CABG procedure, or in any surgical procedure involving an extracorporeal blood circuit.

Also disclosed is the use of a boronic acid compound described herein for the manufacture of a medicament for preventing thrombosis, particularly in an extracorporeal blood circuit during surgery. In embodiments, the medicament is for parenteral, particularly intravenous, administration. In an alternative, the medicament may be administered directly into the extracorporeal blood stream of a patient undergoing a surgical procedure.

In one embodiment, a boronic acid compound of the disclosure is used in the manufacture of a medicament for preventing unwanted coagulation during CABG. In another embodiment, a boronic acid compound which is a free acid, an acid addition salt or a prodrug (whether or not in salt form) is used in the manufacture of a medicament for preventing unwanted coagulation during surgery, for example in surgery involving an extracorporeal blood circuit.

The disclosure includes cardiopulmonary bypass circuits coated (or otherwise associated, e.g. embedded) with a boronic acid compound described herein.

The disclosure further relates to a method of administering a disclosed compound during a surgical procedure involving an extracorporeal blood circuit, of which a CABG procedure with extracorporeal blood circuit is an example. The method comprises the step of administering a therapeutically effective amount of the compound. The compound may either be administered directly into the extracorporeal blood flow or be administered directly to the patient, e.g. by way of intravenous infusion.

The examples of this specification indicate that plasma concentrations of the active principle return to tolerably low levels within about 30 minutes of termination of intravenous administration.

The boronic acids with which the disclosure is concerned are thrombin inhibitors and are, for example, of formula (I), and their salts, prodrugs and prodrug salts:

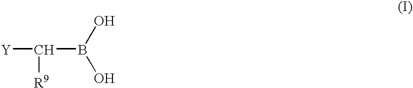

wherein
Y comprises a moiety which, together with the fragment —CH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

In some embodiments, Y comprises

an amino group bonded to structural fragment —CH(R⁹)—B(OH)₂, and

a hydrophobic moiety which is linked to said amino group and which, together with said structural fragment, has affinity for the substrate binding site of thrombin.

The compounds are described in more detail below with particular reference to base addition salts. However, the methods, uses and products disclosed herein are not limited to the salts disclosed below but may use any boronic acid disclosed herein, or any salt or prodrug thereof (the prodrugs themselves optionally being in the form of a salt of a prodrug). It goes without saying that the salts, like he prodrugs, are pharmaceutically acceptable. In general terms, prodrugs may be boronic acid derivatives capable of hydrolysing to release the free boronic acid. As prodrugs may be mentioned esters, e.g. with a residue of an alkanol, e.g. a C₁-C₄ alkanol such as methanol or ethanol, for example. Also to be mentioned are cyclic derivatives, in which the two available valencies of the boron (corresponding to the bonds to the two —OH groups of the free acid) are bonded to respective ends of a chain of atoms, i.e. the boron becomes part of a ring. Such cyclic derivatives may be represented as below in the case of acids of Formula (I), modified mutatis mutandis for acids of other formulae disclosed herein:

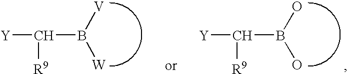

where V and W are heteroatoms (e.g. selected independently from N, O and S) and the arcuate line represents a linear or branched chain of atoms, the length of the chain between the two bonds from the boron is not critical but may be 4, 5 or 6 in some cases. As described, the chain terminated at both ends by the boron (the ring-forming chain) may be linear or branched, e.g., it may have one or more side branches; where there are multiple side branches, at least some of them may join together to form a ring, as in the case of pinanediol esters, for example.

Particular cyclic derivatives, therefore, are cyclic esters formed by diols. The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexyl-ethanediol.

The prodrug may be a sugar derivative as described in WO 02/059131 and equivalent U.S. Pat. No. 6,699,835 (see above). Thus, the boronate group may be esterified with a sugar such as a monosaccharide or a disaccharide, for example. The sugar may be a reduced sugar, e.g. manittol or sorbitol: it may be an individual sugar or class of sugars taught in WO 02/059131. The boronic acid, sugar (or other diol) and water may be combined and then lyophilised, for example as taught in WO 02/059131.

Salts may be acid addition salts or base addition salts.

In one aspect, the disclosure relates to base addition salts of boronic acids which have a neutral aminoboronic acid residue capable of binding to the thrombin S1 subsite linked through a peptide linkage to a hydrophobic moiety capable of binding to the thrombin S2 and S3 subsites. As an example, there is disclosed a parenteral pharmaceutical formulation that includes a pharmaceutically acceptable base addition salt of a boronic acid of, for example, Formula (II):

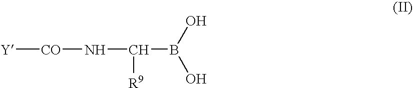

wherein
Y′ comprises a hydrophobic moiety which, together with the aminoboronic acid residue —NHCH(R⁹)—B(OH)₂, has affinity for the substrate binding site of thrombin; and R⁹ is as described before.

The disclosure includes products comprising active ingredients which are base addition salts of hydrophobic boronic acid inhibitors of thrombin. Such inhibitors may contain hydrophobic amino acids, and this class of amino acids includes those whose side chain is hydrocarbyl, hydrocarbyl containing an in-chain oxygen and/or linked to the remainder of the molecule by an in-chain oxygen or heteroaryl, or any of the aforesaid groups when substituted by hydroxy, halogen or trifluoromethyl. Representative hydrophobic side chains include alkyl, alkoxyalkyl, either of the aforesaid when substituted by at least one aryl or heteroaryl, aryl, heteroaryl, aryl substituted by at least one alkyl and heteroaryl substituted by at least one alkyl. Proline and other imino acids which are ring-substituted by nothing or by one of the moieties listed in the previous sentence are also hydrophobic.

Some hydrophobic side chains contain from 1 to 20 carbon atoms, e.g. non-cyclic moieties having 1, 2, 3 or 4 carbon atoms. Side chains comprising a cyclic group typically but not necessarily contain from 5 to 13 ring members and in many cases are phenyl or alkyl substituted by one or two phenyls.

Included are inhibitors which contain hydrophobic non-peptide moieties, which are typically based on moieties which may form a side chain of a hydrophobic amino acid, as described above.

Hydrophobic compounds may contain, for example, one amino group and/or one acid group (e.g. —COOH, —B(OH)₂). Generally, they do not contain multiple polar groups of any one type.

The disclosure comprises products, methods and uses involving or including hydrophobic boronic acid inhibitors of thrombin or their salts or prodrugs, and therefore includes peptide boronic acids which have a partition coefficient between 1-n-octanol and water expressed as log P of greater than 1.0 at physiological pH and 25° C., and the salts and prodrugs thereof. Some useful peptide boronic acids have a partition coefficient of at least 1.5. A class of useful hydrophobic peptide boronic acids has a partition coefficient of no more than 5.

There is a debate in the literature as to whether boronates in aqueous solution form the ‘trigonal’ B(OH)₂ or ‘tetrahedral’ B(OH)₃⁻ boron species, but NMR evidence seems to indicate that at a pH below the first pKa of the boronic acid the main boron species is the neutral B(OH)₂. In the duodenum the pH is likely to be between 6 and 7, so the trigonal species is likely to be predominant here. In any event, the symbol —B(OH)₂ includes tetrahedral as well as trigonal boron species, and throughout this specification symbols indicating trigonal boron species embrace also tetrahedral species. The symbol may further include boronic groups in anhydride form.

The compounds, e.g. salts may be in the form of solvates, particularly hydrates.

The base addition salts may comprise, or consist essentially of, acid salts in which the boronic acid is singly deprotonated. The disclosure therefore includes products having a metal/boronate stoichiometry consistent with the boronate groups in the product predominantly (more than 50 mol %) carrying a single negative charge.

The salts may have a purity, e.g. as determined by the method of Example 33, of at least about 90%, e.g. of greater than or equal to about 95%. In the case of pharmaceutical formulations, such salt forms may be combined with pharmaceutically acceptable diluents, excipients or carriers.

TRI 50c salts may be obtained via TRI 50c esters. However, published synthetic routes to TRI 50c esters and thus to TRI 50c give rise to one or more impurities. The methods described below under the heading ‘“High Purity” synthesis’ (unpublished as of the priority date of this application) for making the salts give rise to one or more impurities and very high purity salts were not obtained. Further, the salts have proved most challenging to obtain in high purity. Thus, purification techniques which were applied failed to produce very high purity salts. HPLC will not be usable on an industrial scale to purify salts made via published TRI 50c ester syntheses and the salt preparation techniques described under the heading ‘“High Purity” synthesis’. In other words, in order for the therapeutic benefits of TRI 50c salts to be provided to those in need thereof, the salts must be obtainable industrially in adequately pure form and the pure form must be attainable without the use of excessively expensive purification techniques.

Parenteral formulations of the salts are also provided herein. In particular, there are provided intravenous formulations, i.e. formulations suitable for intravenous administration, comprising the active ingredient (e.g. base addition salt) in the solid phase, as for example in the case of particulate material (e.g. comprising a base addition salt) for reconstitution as aqueous solutions prior to administration by injection or infusion. Such reconstituted solutions are also included in the disclosure.

According to a further aspect of the present disclosure there is provided a method of treatment of a condition where anti-thrombotic activity is required which method comprises administration of a therapeutically effective amount of a pharmaceutically acceptable base addition salt of a boronic acid described herein, e.g. of Formula (II), to an extracorporeal blood stream, for example, during surgery.

Further aspects and embodiments of the disclosure are described and claimed in the following specification.

The salts described herein include products obtainable by (having the characteristics of a product obtained by) reaction of the boronic acid with a strong base and the term “salt” herein is to be understood accordingly. The term “salt” in relation to the disclosed products, therefore, does not necessarily imply that the products contain discrete cations and anions and is to be understood as embracing products which are obtainable using a reaction of a boronic acid and a base. The disclosure embraces products which, to a greater or lesser extent, are in the form of a coordination compound. The disclosure thus provides also products obtainable by (having the characteristics of a product obtained by) reaction of a disclosed boronic acid with a strong base as well as the therapeutic, including prophylactic, use of such products.

The present disclosure is not limited as to the method of preparation of the salts, provided that they contain a boronate species derived from a disclosed boronic acid and a counter-ion. Such boronate species may be boronate anions in any equilibrium form thereof. The term “equilibrium form” refers to differing forms of the same compounds which may be represented in an equilibrium equation (e.g. boronic acid in equilibrium with a boronic anhydride and in equilibrium with different boronate ions). Boronates in the solid phase may form anhydrides and the disclosed boronate salts when in the solid phase may comprise boronate anhydrides, as a boronic equilibrium species. It is not required that the salts be prepared by reaction of a base containing the counter-ion and the boronic acid. Further, the disclosure includes salt products which might be regarded as indirectly prepared by such an acid/base reaction as well as salts obtainable by (having the characteristics of products obtained by) such indirect preparation. As examples of possibly indirect preparation may be mentioned processes in which, after initial recovery of the salt, it is purified and/or treated to modify its physicochemical properties, for example to modify solid form or hydrate form, or both.

In some embodiments, the cations of the salts are monovalent.

In some embodiments the salts comprise anhydride species; in others they are essentially free of anhydride species.

Further aspects and embodiments of the disclosure are set forth in the following description and claims. Also included as such are the salts described herein.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

This patent application contains data indicating that the stability (resistance to deboronation) of organoboronic acids may be increased by providing them in the form of salts, e.g. metal salts. In single experiments, the ammonium salt of TRI 50c appeared to decompose on drying to yield ammonia, whilst the choline salt demonstrated rapid decomposition to a deboronated impurity. Although experiments have not been conducted to reproduce these unrepeated observations, there is provided a sub-class in which the ammonium and choline salts are excluded. The salt may be an acid salt. In any event, this stabilisation technique forms part of the disclosure and is applicable, inter alia, to organoboronic acids described under the heading “BACKGROUND” and to organoboronic acids described in publications mentioned under that heading.

### DETAILED DESCRIPTION OF SEVERAL EXAMPLES

### Glossary

The following terms and abbreviations are used in this specification:

The expression “acid salt” as applied to a salt of a boronic acid refers to salts of which a single —OH group of the trigonally-represented acid group —B(OH)₂ is deprotonated. Thus salts wherein the boronate group carries a single negative charge and may be represented as —B(OH)(O⁻) or as [—B(OH)₃]⁻ are acid salts. The expression encompasses salts of a cation having a valency n wherein the molar ratio of boronic acid to cation is approximately n to 1. In practical terms, the observed stoichiometry is unlikely to be exactly n:1 but will be consistent with a notional n:1 stoichiometry. For example, the observed mass of the cation might vary from the calculated mass for a n:1 stoichiometry by no more than about 10%, e.g. no more than about 7.5%; in some cases an observed mass of a cation might vary from the calculated mass by no more than about 1%.

Calculated masses are suitably based on the trigonal form of the boronate. (At an atomic level, a salt stoichiometrically consistent with being an acid salt might contain boronates in a mix of protonation states, whose average approximates to single deprotonation and such “mixed” salts are included in the term “acid salt”). Examples of acid salts are monosodium salts and hemicalcium salts.

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids
A=Ala=alanine
V=Val=valine
I=Ile=isoleucine
L=Leu=leucine
M=Met=methionine
F=Phe=phenylalanine
P=Pro=proline
W=Trp=tryptophan

Polar (Neutral or Uncharged) Amino Acids
N=Asn=asparagine
C=Cys=cysteine
Q=Gln=glutamine
G=Gly=glycine
S=Ser=serine
T=Thr=threonine
Y=Tyr=tyrosine

Positively Charged (Basic) Amino Acids
R=Arg=arginine
H=His=histidine
K=Lys=lysine

Negatively Charged Amino Acids
D=Asp=aspartic acid
E=Glu=glutamic acid.
   ACN=acetonitrile
   Amino acid=α-amino acid
   Acid addition salt=a salt which is prepared from addition of an inorganic acid or an organic acid to a free base (e.g. an amino group, as for example an N-terminal amino group of a peptide).
   Base addition salt=a salt which is prepared from addition of an inorganic base or an organic base to a free acid (in this case the boronic acid).
   Cbz=benzyloxycarbonyl
   Cha=cyclohexylalanine (a hydrophobic unnatural amino acid)
   Charged (as applied to drugs or fragments of drug molecules, e.g. amino acid residues)=carrying a charge at physiological pH, as in the case of an amino, amidino or carboxy group
   Dcha=dicyclohexylalanine (a hydrophobic unnatural amino acid)
   Dpa=diphenylalanine (a hydrophobic unnatural amino acid)
   Drug=a pharmaceutically useful substance, whether the active in vivo principle or a prodrug
   Mpg=3-methoxypropylglycine (a hydrophobic unnatural amino acid)
   Multivalent=valency of at least two, for example two or three
   Neutral (as applied to drugs or fragments of drug molecules, e.g. amino acid residues)=uncharged=not carrying a charge at physiological pH
   Pinac=Pinacol=2,3-dimethyl-2,3-butanediol
   Pinanediol=2,3-pinanediol=2,6,6-trimethylbicyclo [3.1.1]heptane-2,3-diol
   Pip=pipecolinic acid
   Room temperature=25° C.+2° C.
   Strong base=a base having a sufficiently high pKb to react with a boronic acid. Suitably such bases have a pKb of 7 or more, e.g. 7.5 or more, for example about 8 or more
   THF=tetrahydrofuran
   Thr=thrombin

### Conversion Factors

Unless otherwise stated the following conversion factors are used in this specification to convert between moles and mass in grams and for other similar calculations:
the molecular weight of TRI 50c is determined in relation to the trigonal form (molecular weight 525.4)
the molecular weight of TRI 50c monosodium salt is calculated on the basis of the monohydrate (C₂₇H₃₅BN₃O₇)Na H₂O (molecular weight 565.39).

### The Compounds

The disclosure relates to boronic acids which have a neutral aminoboronic acid residue capable of binding to the thrombin S1 subsite linked to a hydrophobic moiety capable of binding to the thrombin S2 and S3 subsites. It relates also to prodrugs (e.g. esters) and salts of such acids, particularly base addition salts. The disclosure includes acids of formula (I) above and also those of a sub-class represented by the following formula (II):

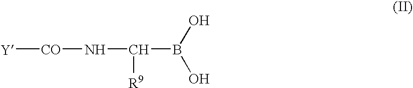

wherein
Y′ comprises a hydrophobic moiety and Y′CO—, together with fragment —NHCR(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 (e.g. 5) or R⁹ is —CH₂)ₘ—W where m is from 2, 3, 4 or 5 (e.g. 4) and W is —OH or halogen (F, Cl, Br or I). As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

Reverting now to formula (I), typically, Y— comprises an amino acid residue (whether natural or unnatural) which binds to the S2 subsite of thrombin, the amino acid residue being N-terminally linked to a moiety which binds the S3 subsite of thrombin. Y— may be of the formula Z³-Z²-CO— where -Z²-CO— is an amino acid residue having affinity for the 52 subsite of thrombin and Z³ is a moiety which has affinity for the 53 subsite of thrombin.

The boronic acid may comprise linkages between the structural fragment —CH(R⁹)—B(OH)₂ and moiety Y or linkages and/or a linkage within Y, e.g. the Z³-Z² linkage, which comprises a nitrogen atom as —NH— or as —NR¹⁴— where R¹⁴ is a C₁-C₁₃ hydrocarbyl group optionally containing in-chain and/or in-ring nitrogen, oxygen or sulfur and optionally substituted by a substituent selected from halo, e.g. F, or a functional group, for example hydroxy. The hydrocarbyl group may contain from 1 to 4 carbon atoms; it may be alkyl or otherwise comprise a moiety bonded to said nitrogen atom which is selected from —CH₂— and halogenated variants thereof, especially fluorinated variants for example —CF₂—.

In one class of Formula (I) acids, Y— is an optionally N-terminally protected dipeptide residue which binds to the 53 and 52 binding sites of thrombin and the peptide linkages in the acid are optionally and independently N-substituted by an R¹⁴ group. The N-terminal protecting group, when present, may be a group X as defined above (other than hydrogen). In many instances, the acid contains no N-substituted peptide linkages; where there is an N-substituted peptide linkage, the substituent is often 1C to 6C hydrocarbyl, e.g. saturated hydrocarbyl; the N-substituent comprises a ring in some embodiments, e.g. cycloalkyl, and may be cyclopentyl, for example. One class of acids has an N-terminal protecting group (e.g. an X group) and unsubstituted peptide linkages.

Where Y— is a dipeptide residue (whether or not N-terminally protected), the S3-binding amino acid residue may be of (R)-configuration and/or the S2-binding residue may of (S)-configuration. The fragment —NHCH(R⁹)—B(OH) may be of ( )-configuration. The disclosure is not restricted to chiral centres of these conformations, however.

In one class of compounds, the side chain of the P3 (S3-binding) amino acid and/or the P2 (S2-binding) amino acid is a moiety other than hydrogen selected from a group of formula A or B:

—(CO)ₐ—(CH₂)_{b}-D_{c}-(CH₂)_{d}-E (A)

—(CO)ₐ—(CH₂)_{b}-D_{c}-Cₑ(E¹)(E²)(E³) (B)

wherein
a is 0 or 1;
e is 1;
b and d are independently 0 or an integer such that (b+d) is from 0 to 4 or, as the case may be, (b+e) is from 1 to 4;
c is 0 or 1;

### D is O or S;

E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)ᵣ and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and
E¹, E² and E³ are each independently selected from —R¹⁵ and -J-R¹⁵, where J is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -J-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1); in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of embodiments, E contains a substituent which is C₁-C₆ alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of embodiments, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

The disclosure includes salts in which the P3 and/or P2 side chain comprises a cyclic group in which 1 or 2 hydrogens have been replaced by halogen, e.g. F or Cl.

The disclosure includes a class of salts in which the side chains of formula (A) or (B) are of the following formulae (i), (ii) or (iii), or be variants thereof in which one or both phenyl rings of (ii) or the phenyl ring of (iii) are replaced by cyclohexyl or cyclohexenyl:

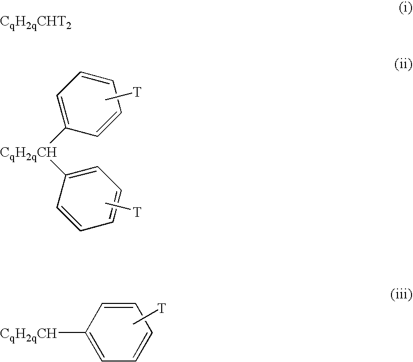

wherein q is from 0 to 5, e.g. is 0, 1 or 2, and each T is independently hydrogen, 1, 2 or 3 halogens (e.g. F or Cl), —SiMe₃, —CN, —R¹³, —OR¹³, —COR¹³, —CO₂R¹³ or —O₂CR¹³. In some embodiments of structures (ii) and (iii), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (I) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

In many Y— groups which are dipeptide fragments (which dipeptides may be N-terminally protected or not), the P3 amino acid has a side chain of formula (A) or (B) as described above and the P2 residue is of an imino acid.

The disclosure therefore includes medicaments comprising base addition salts, e.g. metal salts, of organoboronic acids which are thrombin inhibitors, particularly selective thrombin inhibitors, having a neutral P1 (S1-binding) moiety. For more information about moieties which bind to the S3, S2 and Si sites of thrombin, see for example Tapparelli C et al, Trends Pharmacol. Sci 14: 366-376, 1993; Sanderson P et al, Current Medicinal Chemistry, 5: 289-304, 1998; Rewinkel 3 et al, Current Pharmaceutical Design, 5:1043-1075, 1999; and Coburn C Exp. Opin. Ther. Patents 11(5): 721-738, 2001. The thrombin inhibitory salts of the disclosure are not limited to those having S3, S2 and S1 affinity groups described in the publications listed in the preceding sentence. Alternatively to being presented as a base addition salt, the organoboronic acids may be presented as the free acid, an acid addition salt or a prodrug (e.g. ester).

The boronic acids may have a Ki for thrombin of about 100 nM or less, e.g. about 20 nM or less.

A subset of the Formula (I) acids comprises the acids of Formula (III):

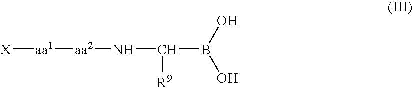

X is a moiety bonded to the N-terminal amino group and may be H to form NH₂. The identity of X is not critical but may be a particular X moiety described above. In one example there may be mentioned benzyloxycarbonyl.

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0, 1 and 2 are preferred) and R⁶ is H or a 5 to 13-membered cyclic group optionally substituted by one or more (e.g. 1, 2, 3, 4 or 5) halogens (e.g. F), for example at least at the 4-position, and/or by 1, 2 or 3 substituents selected from amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group.

More particularly X is R⁶—(CH₂)ₚ—C(O)— or R⁶—(CH₂)ₚ—O—C(O)— and p is 0, 1 or 2 in these moieties, R⁶ may be phenyl or fluorophenyl. Said 5 to 13-membered cyclic group is often aromatic or heteroaromatic, for example is a 6-membered aromatic or heteroaromatic group. In many cases, the group is not substituted.

Exemplary X groups are (2-pyrazine) carbonyl, (2-pyrazine) sulfonyl and particularly benzyloxycarbonyl or benzylmethylcarbonyl.

aa¹ is an amino acid residue having a hydrocarbyl side chain containing no more than 20 carbon atoms (e.g. up to 15 and optionally up to 13 C atoms) and comprising at least one cyclic group having up to 13 carbon atoms. In certain examples, the cyclic group(s) of aa¹ have/has 5 or 6 ring members. For instance, the cyclic group(s) of aa¹ may be aryl groups, particularly phenyl. Typically, there are one or two cyclic groups in the aa¹ side chain. Certain side chains comprise, or consist of, methyl substituted by one or two 5- or 6-membered rings.

More particularly, aa¹ is Phe, Dpa or a wholly or partially hydrogenated analogue thereof. The wholly hydrogenated analogues are Cha and Dcha.

aa² is an imino acid residue having from 4 to 6 ring members. Alternatively, aa² is Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

As another alternative, aa² is the β-amino acid analogue of Gly (i.e. H₂N—CH₂—CH₂—COOH) N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

The disclosure includes a class of compounds in which aa² is a residue of a β-amino acid having a 4 to 6 membered carbocyclic ring which optionally has one carbon atom replaced by a sulfur and of which the ring-forming carbon atoms include the carbon atoms α- and β- to the carboxyl group (i.e. the β-amino acid comprises a 4 to 6 membered carbocyclic ring which is 1-substituted by carboxyl and 2-substituted by amino and which may at one other position contain an S atom.

An exemplary class of products comprises those in which aa² is a residue of an imino acid of formula (IV)

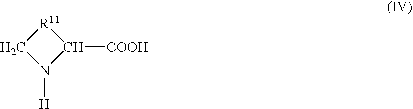

where R¹¹ is —CH₂—, —CH₂—CH₂—, —CH═CH—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline are illustrative.

Also to be mentioned as aa² are β-amino acids of formula (XXVI):

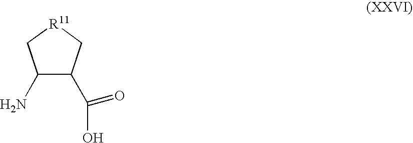

wherein R¹¹ is as previously defined.

In embodiments, aa² is a residue of an N-substituted imino acid or β-amino acid.

It will be appreciated from the above that a very preferred class of products consists of those in which aa¹-aa² is Phe-Pro. In another preferred class, aa¹-aa² is Dpa-Pro. In other products, aa¹-aa² is Cha-Pro or Dcha-Pro. Of course, also included are corresponding product classes in which Pro is replaced by (s)-azetidine-2-carboxylic acid.

R⁹ is as defined previously and may be a moiety R¹ of the formula —(CH₂)ₛ-Z. Integer s is 2, 3 or 4 and W is —OH, —OMe, —OEt or halogen (F, Cl, I or, preferably, Br). Particularly illustrative Z groups are —OMe and —OEt, especially —OMe. In certain examples s is 3 for all Z groups and, indeed, for all compounds of the disclosure. Particular R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

Accordingly, a specific class of acids are those of the formula X-Phe-Pro-Mpg-B(OH)₂, especially Cbz-Phe-Pro-Mpg-B(OH)₂; also included are analogues of these compounds in which Mpg is replaced by a residue with another of the R¹ groups and/or Phe is replaced by Dpa or another aa¹ residue. Also included are compounds in which Cbz is replaced by benzylmethylcarbonyl (Ph-Et-CO—).

The aa¹ moiety of the acid is preferably of R configuration. The aa² moiety is preferably of (S)-configuration. Particularly preferred compounds have aa¹ of (R)-configuration and aa² of (S)-configuration. The chiral centre —NH—CH(R¹)—B— is preferably of (R)-configuration. It is considered that commercial formulations will have the chiral centres in (R,S,R) arrangement, as for example in the case of salts of Cbz-Phe-Pro-BoroMpg-OH:

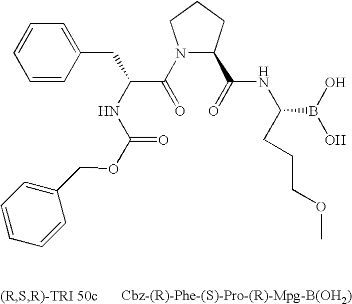

In preferred embodiments, the various aspects of the disclosure relate to pharmaceutically acceptable base addition salts of the described acids.

The disclosure includes base addition salts of Cbz-(R)-Phe-(S)-Pro-(R)-boroMpg-OH (and of other compounds of the formula X—(R)-Phe-(S)-Pro-(R)-boroMpg-OH) which are at least 90% pure, e.g. at least 95% pure.

In broad terms, the base addition salts described herein may be considered to correspond to reaction products of an organoboronic acid as described above with a strong base, e.g. a basic metal compound; the salts are however not limited to products resulting from such a reaction and may be obtained by alternative routes.

The base addition salts are therefore obtainable by contacting a boronic acid disclosed herein with a strong base. The disclosure thus contemplates products (compositions of matter) having the characteristics of a reaction product of an acid of formula (I) and a strong base. The base is pharmaceutically acceptable.

As suitable salts may be mentioned salts of metals, e.g. of monovalent or divalent metals, and stronger organic bases, for example:

1. Alkali metal salts;
2. Divalent, e.g. alkaline earth metal, salts;
3. Group III metals;
4. Salts of strongly basic organic nitrogen-containing compounds, including:
4A. Salts of guanidines and their analogues;
4B. Salts of strongly basic amine, examples of which include (i) aminosugars and (ii) other amines.

Of the above salts, particularly illustrative are alkali metals, especially Na and Li. Also illustrative are aminosugars.

Specific salts are of the acid boronate though in practice the acid salts may contain a very small proportion of the doubly deprotonated boronate. The term “acid boronate” refers to trigonal —B(OH)₂ groups in which one of the B—OH groups is deprotonated as well as to corresponding tetrahedral groups in equilibrium therewith. Acid boronates have a stoichiometry consistent with single deprotonation.

The disclosure includes therefore products (compositions of matter) which comprise salts which may be represented by formula (V):

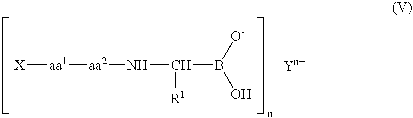

where Yⁿ⁺ is a pharmaceutically acceptable cation obtainable from a strong base, and aa¹, aa², X and R¹ are as defined above. Also included are products in which R¹ is replaced by another R⁹ group.

One class of salts have a solubility of about 10 mM or more, e.g. of at least about 20 mM, when their solubility is determined as described in the examples at a dissolution of 25 mg/ml. More particularly yet they have a solubility of least 50 mM when their solubility is determined as described in the examples at a dissolution of 50 mg/ml.

The disclosure includes salts of boronic acids (I) having an observed stoichiometry consistent with the salt being of (being representable by) the formula “(boronate⁻)ₙ cationⁿ⁺”. One class of such salts are represented by the formula:

[Cbz-(R)-Phe-(S)-Pro-(R)-Mpg-B(OH)(O⁻)]M⁺

where M⁺ represents a monovalent cation, especially an alkali metal cation. It will be understood that the above representation is a notional representation of a product whose observed stoichiometry is unlikely to be literally and exactly 1:1. In any event, a particular salt is Cbz-(R)-Phe-(S)-Pro-(R)-Mpg-B(OH)₂ monosodium salt (TGN 255). In the above formula, the trigonally-represented boronate represents, as always, boronates which are trigonal, tetrahedral or mixed trigonal/tetrahedral.

Particularly exemplary are products which comprise:

(i) species selected from (a) acids of formula (VIII): X—(R)-Phe-(S)-Pro-(R)-Mpg-B(OH)₂ where X is H or an amino-protecting group, especially Cbz, (b) boronate anions thereof, and (c) any equilibrium form of the aforegoing (e.g. an anhydride); and
(ii) ions having a valency n in combination with said species, the species and said ions having an observed stoichiometry consistent with a notional species:ion stoichiometry of n:1. In one class of salts, n is 1.

Considering the counter-ions in turn:

### 1. Monovalent Metal, Especially Alkali Metal Salts

Suitable alkali metals include lithium, sodium and potassium. All of these are remarkably soluble. Lithium and sodium are illustrative because of their high solubility. The lithium and particularly sodium salts are of surprisingly high solubility in relation to potassium amongst others. Sodium is most used in many instances. Salts containing mixtures of alkali metals are contemplated by the disclosure.

The disclosure includes products comprising salts of the formula (VI)

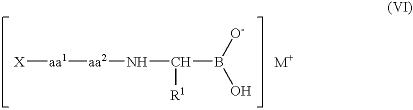

where M⁺ is an alkali metal ion and aa¹, aa², X and R¹ are as defined above, as well as salts in which both hydroxy groups of the boronate group are in salt form (preferably with another identical M⁺ group) and mixtures of such salts. Included also are products wherein R¹ is replaced by another R⁹ group.
2. Divalent, e.g. Alkaline Earth Metal (Group II Metal) Salts

One example of a divalent metal is calcium. Another suitable divalent metal is magnesium. Also contemplated is zinc. The divalent metals are usually used in a boronic acid:metal ratio of substantially 2:1, in order to achieve the preferred monovalent boronate moiety. Salts containing mixtures of divalent metals, e.g. mixtures of alkaline earth metals, are also contemplated.

Further disclosed are products (compositions of matter) which comprise salts which may be represented by the formula (VII):

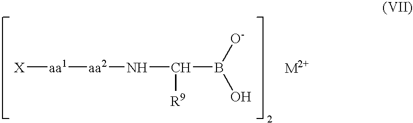

where M²⁺ is a divalent metal cation, e.g. an alkaline earth metal or zinc cation, and aa¹, aa², X and R⁹ are as defined above, as well as salts in which both hydroxy groups of the boronate group are deprotonated and mixtures of such salts. As previously indicated, the boronate may comprise a tetrahedral species.

### 3. Group III Metals

Suitable Group III metals include aluminium and gallium. Salts containing mixtures of Group III metals are also contemplated.

The disclosure includes products comprising salts of the formula (VIII):

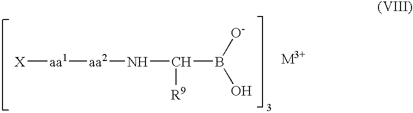

where M³⁺ is a Group III metal ion and aa¹, aa², X and R⁹ are as defined above, as well as salts in which both hydroxy groups of the boronate group are in salt form and mixtures of such salts. As previously indicated, the boronate may comprise a tetrahedral species.

### 4. Strongly Basic Organic Nitrogen-Containing Compounds

The disclosure includes products obtainable by (having the characteristics of a product obtained by) reaction of a peptide boronic acid as defined above and a strong organic base. Two illustrative classes of organic base are described in sections 4A and 4B below. Particularly preferred are acid salts (in which one of the two boronic —OH groups is deprotonated). Most commonly, the salts contain a single type of organic counter-ion (disregarding trace contaminants) but the disclosure contemplates salts containing mixtures of organic counter-ions; in one sub-class, the different counter-ions all fall within the section 4A family described below or, as the case may be, in the section 2B family below; in another subclass, the salts comprise a mixture of organic counter-ions which are not all from the same family (4A or 4B).

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base.

4A. Guanidines and their Analogues

The guanidino compound (guanidine) may in principle be any soluble and pharmaceutically acceptable compound having a guanidino or a substituted guanidino group, or a substituted or unsubstituted guanidine analogue. Suitable substituents include aryl (e.g. phenyl), alkyl or alkyl interrupted by an ether or thioether linkage and, in any event, typically contain from 1 to 6 and especially 1, 2 3, or 4 carbon atoms, as in the case of methyl or ethyl. The guanidino group may have 1, 2, 3 or 4 substituent groups but more usually has 1 or 2 substituent groups, for instance on a terminal nitrogen. One class of guanidines is monoalkylated; another class is dialkylated. As guanidine analogues may be mentioned thioguanidines and 2-amino pyridines. Compounds having unsubstituted guanidino groups, for example guanidine and arginine, form one particular class.

Salts containing mixtures of guanidines are contemplated by the disclosure.

A particular guanidino compound is L-arginine or an L-arginine analogue, for example D-arginine, or the D- or, preferably, L-isomers of homoarginine or agmatine [(4-aminobutyl)guanidine]. Less preferred arginine analogues are NG-nitro-L-arginine methyl ester, for example, and constrained guanidine analogues, particularly 2-amino pyrimidines, for example 2,6-quinazolinediamines such as 5,6,7,8-tetrahydro-2,6-quinazolinediamine, for example. The guanidino compound may also be a peptide, for example a dipeptide, containing arginine; one such dipeptide is L-tyrosyl-L-arginine.

Some particular guanidino compounds are compounds of formula (VII):

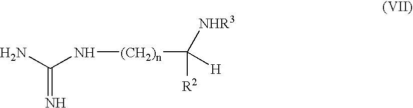

where n is from 1 to 6 and for example at least 2, e.g. 3 or more, and in many instances no more than 5. Most particularly, n is 3, 4 or 5. R² is H or carboxylate or derivatised carboxylate, for example to form an ester (e.g. a C₁-C₄ alkyl ester) or amide. R³ is H, C₁-C₄ alkyl or a residue of a natural or unnatural amino acid (e.g. tyrosine). The compounds of formula (IV) are usually of L-configuration. The compounds of formula (IV) are arginine (n=3; R²=carboxyl; R³=H) and arginine derivatives or analogues.

The disclosure includes products comprising salts of the formula (IX)

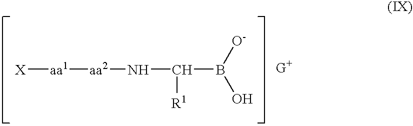

where aa¹ aa², X and R¹ are as defined previously and G⁺ is the protonated form of a pharmaceutically acceptable organic compound comprising a guanidino group or an analogue thereof, as well as salts in which both hydroxy groups of the boronate group are in salt form (preferably with another identical G+group) and mixtures of such salts. Also included are products wherein R¹ is replaced by another R⁹ group.

### 4B. Strongly Basic Amines

The disclosure includes products obtainable by (having the characteristics of a product obtained by) reaction of a peptide boronic acid as defined above and a strong organic base which is an amine. The amine may in principle be any soluble and pharmaceutically acceptable amine.

It is envisaged that a desirable class of amine includes those having polar functional groups in addition to a single amine group, as such compounds will be more hydrophilic and thus more soluble than others. In certain salts, the or each additional functional group is hydroxy. Some amines have 1, 2, 3, 4, 5 or 6 additional functional groups, especially hydroxy groups. In one illustrative class of amines the ratio of (amino plus hydroxy groups):carbon atoms is from 1:2 to 1:1, the latter ratio being particularly preferred. These amines with one or more additional polar functional groups may be a hydrocarbon, especially an alkane, substituted by the amino group and the additional polar group(s). The amino group may be substituted or unsubstituted and, excluding amino substituents, the polar base may contain, for example, up to 10 carbon atoms; usually there are no less than three such carbon atoms, e.g. 4, 5 or 6. Aminosugars are included in this category of polar bases.

The disclosure includes products comprising salts of the formula (X)

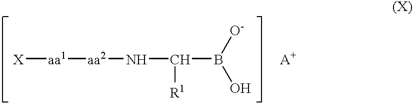

where aa¹, aa², X and R¹ are as defined previously and A⁺ is the protonated form of a pharmaceutically acceptable amine, as well as salts in which both hydroxy groups of the boronate group are in salt form (preferably with another identical A⁺ group) and mixtures of such salts. In one class of such products, A⁺ is the protonated form of an amine described in section 2B(i) below; in another class A⁺ is the protonated form of an amine described in 2B(ii) below. Also included are products in which R¹ is replaced by another R⁹ group.

Two illustrative classes of amine base are described in sections 4B(i) and 4B(ii) below. Particularly preferred are acid salts (in which one of the two boronic OH groups is deprotonated). Most commonly, the salts contain a single type of amine counter-ion (disregarding trace contaminants) but the disclosure contemplates salts containing mixtures of amine counter-ions; in one sub-class, the different counter-ions all fall within the sub-section 4B(i) family described below or, as the case may be, in the sub-section 4B(ii) family below; in another subclass, the salts comprise a mixture of organic counter-ions which are not all from the same family (4B(i) or 4B(ii)).

4B(i) Aminosugars

The identity of the aminosugar is not critical. Preferred aminosugars include ring-opened sugars, especially glucamines. Cyclic aminosugars are also envisaged as useful. One class of the aminosugars is N-unsubstituted and another, preferred, class is N-substituted by one or two N-substituents (e.g. one). Suitable substituents are hydrocarbyl groups, for example and without limitation containing from 1 to 12 carbon atoms; the substituents may comprise alkyl or aryl moieties or both. Exemplary substituents are C₁, C₂, C₃, C₄, C₅, C₆, C₇ and C₈ alkyl groups, in particular methyl and ethyl, of which methyl is illustrative. Data indicate that aminosugars, especially N-methyl-D-glucamine, are of surprisingly high solubility.

A most preferred aminosugar is N-methyl-D-glucamine:

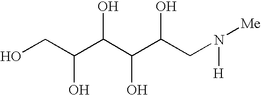

4B(ii) Other Amines

Other suitable amines include amino acids (whether naturally occurring or not) whose side chain is substituted by an amino group, especially lysine.

Some amines are compounds of formula (XI):

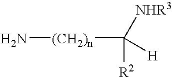

where n, R² and R³ are as defined in relation to formula (IV). The compounds of formula (VI) are usually of L-configuration. The compounds of formula (VI) are lysine (n=4; R²=carboxyl; R³=H) and lysine derivatives or analogues. A most preferred amine is L-lysine.

Other suitable amines are nitrogen-containing heterocycles. At least usually, such heterocyclic compounds are alicyclic; one class of the heterocyclic compounds is N-substituted and another, preferred, class is N-unsubstituted. The heterocycles may contain 6 ring-forming atoms, as in the cases of piperidine, piperazine and morpholine. One class of amines includes N-containing heterocycles substituted by polar substituents, especially hydroxy, e.g. 1, 2 or 3 times.

The disclosure therefore includes amines other than aminosugars which have one or more (e.g. 1, 2, 3, 4, 5 or 6) polar substituents, especially hydroxy, in addition to one amine group. Such compounds may have a ratio of (amino plus hydroxy groups):carbon atoms of 1:2 to 1:1, the latter ratio being particularly preferred.

The disclosure includes mixed salts, i.e. salts containing a mixture of boropeptide moieties and/or counterions but single salts are preferred.

The salts in solid form may contain a solvent, e.g. water. There are included a class of products in which the salts are essentially anhydrous. Also included is a class in which the salts are hydrates.

Also to be mentioned as well as base addition salts are acid addition salts. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate.

### Novel Boronic Acids

The disclosure provides novel boronic acids and derivatives thereof, useful in the treatment, e.g. prevention, of thrombosis. The novel acids include those of the formula (IA):

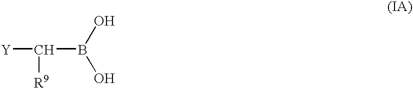

wherein
Y comprises a moiety which, together with the fragment —CH(R⁹)—B(OH)₂, has affinity for the substrate binding site of thrombin and which includes a thrombin P2 domain which comprises a residue of a β-amino acid; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

The β-amino acid has affinity for the S2 subsite of thrombin and may be a β-amino acid or a β-imino acid.

The β-amino acid may be the α-amino acid analogue of Gly (i.e. H₂N—CH₂—CH₂—COOH) N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-diallylphenylethyl.

The disclosure includes a class of compounds in which the β-amino acid is a residue of a β-amino acid having a 4 to 6 membered carbocyclic ring which optionally has one carbon atom replaced by a sulfur and of which the ring-forming carbon atoms include the carbon atoms α- and β- to the carboxyl group (i.e. the β-amino acid comprises a 4 to 6 membered carbocyclic ring which is 1-substituted by carboxyl and 2-substituted by amino and which may at one other position contain an S atom).

Also to be mentioned as the β-amino acid are β-amino acids of formula (XXVI):

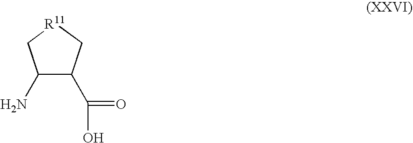

wherein R¹¹ is as previously defined.

In embodiments, the β-amino acid is a residue of an N-substituted imino acid or N-substituted β-amino acid.

Included in the disclosure are acids of formula (II) above in which aa² is a β-amino acid as disclosed herein.

The novel acids may be in the form of the acid, a salt, a prodrug or a salt of a prodrug, as disclosed herein previously. They may be an ester, a base addition salt or an acid addition salt, for example. The ester may be of a diol which has previously been mentioned, e.g. pinacol, pinanediol or a sugar, e.g. mannitol or sorbitol.

The acids and their derivatives may be presented as pharmaceutical formulation, either alone or in combination with a pharmaceutically acceptable diluent, excipient or carrier. The disclosure is not restricted as to the type of formulation, it may be for oral administration, e.g. as a tablet, capsule, granules or powder. The formulation may be a reconstitutable formulation. Tablets or capsules may be enterically coated or not.

As parenteral formulations may be mentioned intravenous formulations (e.g. isotonic solutions) or powders/granules for reconstitution as a liquid intavenous formulation. Parenteral formulations may comprise finely divided powder, e.g. freeze dried powder, optionally including a suitable excipient, e.g. isotonic agent.

The compounds may be administered to inhibit thrombin in the treatment of disease, e.g. for an indication described in this specification or any other indication for which thrombin inhibition is beneficial.

Other structural and functional characteristics of embodiments of the new compounds are as described herein in relation to the compounds used in the formulations, methods and uses disclosed herein.

The novel compounds provide a choice. It is contemplated that, at least in embodiments, the compounds will, at least in broad terms, maintain or improve potency or specificity, or both as compared with TRI 50c.

At least in embodiments, the compounds may maintain or enhance bioavailability. Other properties of novel compounds which may lend them pharmaceutical usefulness may include storage stability or ease of formulation, for example.

The synthesis of 2-amino-cycloalkylcarboxylic acids is described in WO 98/03540.

Synthetic Methods and their Produce

### 1. Peptide/Peptidomimetic Synthesis

The synthesis of boropeptides, including, for example, Cbz-D-Phe-Pro-BoroMpg-OPinacol is familiar to those skilled in the art and described in the prior art mentioned above, including Claeson et al (U.S. Pat. No. 5,574,014 and others) and Kakkar et al (WO 92/07869 and family members including U.S. Pat. No. 5,648,338). It is described also by Elgendy et al Adv. Exp. Med. Biol. (USA) 340:173-178, 1993; Claeson, G. et al Biochem. J. 290:309-312, 1993; Deadman et al J. Enzyme Inhibition 9:29-41, 1995, and by Deadman et al J. Med. Chem. 38:1511-1522, 1995.

Stereoselective synthesis with S or R configuration at the chiral B-terminal carbon may be conducted using established methodology (Elgendy et al Tetrahedron. Lett 33:4209-4212, 1992; WO 92/07869 and family members including U.S. Pat. No. 5,648,338) using (+) or (−)-pinanediol as the chiral director (Matteson et al J. Am. Chem. Soc. 108:810-819, 1986; Matteson et al Organometallics. 3:1284-1288, 1984). Another approach is to resolve the requisite aminoboronate intermediate (e.g. Mpg-BOPinacol) to selectively obtain the desired (R)-isomer and couple it to the dipeptide moiety (e.g. Cbz-(R)-Phe-(S)-Pro, which is the same as Cbz-D-Phe-L-Pro) which will form the remainder of the molecule.

The boropeptides may be synthesised initially in the form of boronic acid esters, particularly esters with diols. Such diol esters may be converted to the peptide boronic acid as described next.

### 2. Ester to Acid Conversion

A peptide boronate ester such as Cbz-(R)-Phe-Pro-BoroMpg-OPinacol may be hydrolysed to form the corresponding acid.

A novel technique for converting a diol ester of a peptide boronic acid of for example, formula (I) into the acid comprises dissolving the diol ester in an ether and particularly a dialkyl ether, reacting the thus-dissolved diol with a diolamine, for example a dialkanolamine, to form a product precipitate, recovering the precipitate, dissolving it in a polar organic solvent and reacting the thus-dissolved product with an aqueous medium, e.g. an aqueous acid, to form the peptide boronic acid. The boronic acid may be recovered from the organic layer of the mixture resulting from the reaction, for example by removing the solvent, e.g. by evaporation under vacuum or distillation. The reaction between the diol ester and the diolamine may be carried out under reflux, for example.

The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexylethanediol.

The alkyl groups of the dialkyl ether preferably have 1, 2, 3 or 4 carbon atoms and the alkyl groups may be the same or different. An exemplary ether is diethyl ether.

The alkyl groups of the dialkanolamine preferably have 1, 2, 3 or 4 carbon atoms and the alkyl groups may be the same or different. An exemplary dialkanolamine is diethanolamine. The diethanolamine/boronic acid reaction product hydrolyses in water at room temperature and the rate of hydrolysis may be accelerated by adding acid or base.

The polar organic solvent is preferably CHCl₃. Other examples are polyhalogenated alkanes generally and ethyl acetate. In principle, any polar organic solvent is acceptable other than alcohols.

The aqueous acid is suitably a strong inorganic acid at a pH in the region of 1 such as hydrochloric acid, for example.

After reaction with the acid, the reaction mixture is suitably washed with, for example, NH₄Cl or another mild base.

An example of a specific procedure is as follows

1. The pinacol or pinanediol ester of the selected peptide boronic acid is dissolved in diethylether.

2. Diethanolamine is added and the mixture is refluxed at 40° C.

3. The precipitated product is removed (filtered), washed (usually several times) with diethyl ether or another polar organic solvent other than an alcohol, and dried (e.g. by evaporation under vacuum).

4. The dry product is dissolved in a polar organic solvent other than an alcohol, e.g. CHCl₃. Aqueous acid or base is added, e.g. hydrochloric acid (pH 1), and the mixture is stirred for e.g. approximately 1 h at room temperature.

5. The organic layer is removed and washed with NH₄Cl solution.

6. The organic solvent is distilled off and the residual solid product is dried.

The above process results in the formation of what may conveniently be referred to as a “diolamine adduct” of the peptide boronic acid, especially such adducts with diethanolamine, and such adducts are themselves included in the disclosure.

It will be appreciated that the aforegoing technique comprises an example of a method for recovering an organoboronic acid product, the method comprising providing in a solvent a dissolved mixture comprising the organoboronic acid in a soluble form and a compound having two hydroxy groups and an amino group (i.e. a diolamine), causing or allowing the organoboronic acid and the diolamine to react to form a precipitate, and recovering the precipitate. The soluble form of the organoboronic acid may be a diol ester, as discussed above. The solvent may be an ether, as discussed above. The organoboronic acid may be one of the organoboronic acids referred to in this specification, for example it may be of Formula (II) or (III). The method described in this paragraph is novel and forms an aspect of the disclosure. A recovery method is filtration.

The reaction between the diolamine and the soluble form of the organoboronic acid is suitable carried out at an elevated temperature, for example under reflux.

Another aspect of the disclosure is a method for recovering an organoboron species, comprising

providing, in a form soluble in an ether, an organoboronic acid, for example a drug such as, e.g., a compound of formula (III);

forming a solution of the soluble form in the ether;

combining the solution with a dialkanolamine and allowing or causing the dialkanolamine to react with the soluble form of the organoboronic acid to form an insoluble precipitate; and

recovering the precipitate.

The term “soluble” in the preceding paragraph refers to species which are substantially more soluble in the reaction medium than is the precipitated product. In variants of the method, the ether is replaced by toluene or another aromatic solvent.

The diethanolamine precipitation technique described above is an example of another novel method, which is a method for recovering from ether solution a pinacol or pinanediol ester of a peptide boronic acid, comprising dissolving diethanolamine in the solution, allowing or causing a precipitate to form and recovering the precipitate. The disclosure encompasses variants of this methods in which another diol than pinacol or pinanediol is used.

The precipitated material, i.e. the “adduct”, may be converted into the free organoboronic acid, for example by contacting it with an acid. The acid may be an aqueous acid, for example an aqueous inorganic acid, e.g. as described above. The precipitate may be dissolved, for example in an organic solvent, prior to being contacted with the acid.

The disclosure therefore provides a method for making an organoboronic acid, comprising converting its diolamine reaction product to the acid.

The acid resulting from the methods described in the previous two paragraphs may be converted to a salt of the acid with a multivalent metal, which salt may in turn be formulated into a pharmaceutical composition in parenteral dosage form.

### 3. Salt Synthesis

3.1 Base Addition salts

In general, the base addition salts may be prepared by contacting the relevant peptide boronic acid with a strong base appropriate to form the desired salt. In the case of metal salts, the metal hydroxides are suitable bases (alternatively, metal carbonates might be used, for example), whilst sometimes it is more convenient to contact the acid with a relevant metal alkoxide (e.g. methoxide), for which purpose the corresponding alkanol is a suitable solvent. Salts with organic bases may be prepared by contacting the peptide boronic acid with the organic base itself. Illustrative salts are acid salts (one —BOH proton replaced) and, to make acid salts with a monovalent cation, the acid and the base are suitably reacted in substantially equimolar quantities. Generally stated, therefore, the usual acid:base molar ratio is substantially n:1, where n is the valency of the cation of the base.

In one procedure, a solution of the peptide boronic acid in a water-miscible organic solvent, for example acetonitrile or an alcohol (e.g. ethanol, methanol, a propanol, for example iso-propanol, or another alkanol), is combined with an aqueous solution of the base. The acid and the base are allowed to react and the salt is recovered. The reaction is typically carried out at ambient temperature (e.g. at a temperature of from 15 to 30° C., e.g. 15 to 25° C.), but an elevated temperature may be used, for example up to the boiling point of the reaction mixture but more usually lower, e.g. a temperature of up to 40° C. or 50° C. The reaction mixture may be allowed to stand or be agitated (usually stirred).

The time during which the acid and the base are allowed to react is not critical but it has been found desirable to maintain the reaction mixture for at least one hour. A period of from one to two hours is usually suitable but longer reaction times may be employed.

The salt may be recovered from the reaction mixture by any suitable method, for example evaporation or precipitation. Precipitation may be carried out by adding an excess of a miscible solvent in which the salt has limited solubility. In one preferred technique, the salt is recovered by evacuating the reaction mixture to dryness. The salt is preferably thereafter purified, for example by redissolving the salt before filtering the resulting solution and drying it, for example by evacuating it to dryness. The redissolution may be performed using water, e.g. distilled water. The salt may then be further purified, for example in order to remove residual water by further redissolution in a suitable solvent, which is advantageously ethyl acetate or THF followed by evaporating to dryness. The purification procedure may be carried out at ambient temperature (say, 15 to 30° C., e.g. 15 to 25° C.), or at a modestly elevated temperature, such as e.g. a temperature not exceeding 40° C. or 50° C.; for example the salt may be dissolved in water and/or solvent by agitating with or without warming to, for example, 37° C.

Also included is a method for drying the salts of the disclosure and other peptide boronic acid salts, comprising dissolving them in an organic solvent, e.g. ethyl acetate or THF, and then evaporating to dryness, e.g. by evacuation.

Generally, preferred solvents for use in purifying the salts are ethyl acetate or THF, or perhaps another organic solvent.

A general procedure for synthesising salts of Cbz-Phe-Pro-BoroMpg-OH is as follows:

Cbz-Phe-Pro-BoroMpg-OH (20.00 g, 38.1 mM) is dissolved in acetonitrile (200 ml) with stirring at room temperature. To this solution is added the requisite base in solution in distilled water (190 ml); the base is added as a 0.2M solution for a monovalent cation. The resultant clear solution is allowed to react for example by being left to stand or being agitated, for a usual period, in either case, of from one to two hours. The reaction is typically carried out at ambient temperature (e.g. 15-30° C., e.g. 15 to 25° C.) but alternatively the temperature may be elevated (e.g. up to 30° C., 40° C. or 50° C.). The reaction mixture is then evacuated to dryness under vacuum with its temperature not exceeding 37° C., typically to yield a white brittle solid or an oil/tacky liquid. The oil/tacky liquid is redissolved in the minimum amount of distilled water necessary (200 ml to 4 L), typically with warming (e.g. to 30-40° C.), usually for up to 2 hours. The solution is filtered, suitably through filter paper, and evacuated to dryness, again with the temperature of the solution not exceeding 37° C., or freeze dried. The resultant product is dried under vacuum overnight to normally yield a white brittle solid. If the product is present as an oil or tacky solid then it is dissolved in ethyl acetate and evacuated to dryness to produce the product as a white solid. The white solid is typically a coarse, amorphous powder.

In variations of the aforegoing general procedure, the acetonitrile is replaced by another water-miscible organic solvent, notably an alcohol, as discussed above, especially ethanol, methanol, iso-propanol or another propanol.

Where a boronic acid salt is less soluble in a selected reaction medium for salt formation such that its direct preparation from the corresponding acid and base is inconvenient, the less soluble salt may be prepared from a salt more soluble in the reaction medium.

There is provided also the use of a boronic acid to make a base addition salt of the disclosure. Included also is a method of preparing a product of the disclosure, comprising contacting a boronic acid, e.g. of formula (I), (II) or (III), with a base capable of making such a salt.

### 3.2 Acid Addition Salts

In general, the acid addition salts may be prepared by contacting the relevant boropeptide (e.g. boronic acid or ester or other prodrug) with an acid appropriate to form the desired salt.

### 4. Separation of Stereoisomers

The stereoisomers of a peptide boronic ester or a synthetic intermediate aminoboronate may be resolved in, for example, any known way. In particular, stereoisomers of boronic esters may be resolved by HPLC.

### 5. “High Purity” Synthesis

The literature teaches that organoboronic acids are degraded by oxidation of the C—B bond. See for example Wu et al (see above). Earlier work on the salts of TRI 50c confirmed that these salts and/or intermediates in their preparation are slightly unstable, to the extent that the salts were found to contain a boron-free impurity, designated impurity I, which was evidently generated by C—B bond cleavage. The salts as a class are significantly more stable to such degradation than the free acid.

These earlier TRI 50c salts were made via the general methods described in Examples 5 and 9 of this specification. Impurity I has the following structure:

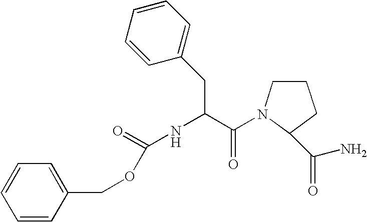

Relative chiral purity of salts made following the general procedure of Examples 5 and 9 was achieved by resolving by HPLC the pinacol ester of TRI 50c, designated TRI 50b, and converting the thus-resolved TRI 50b into the salts. Such an HPLC procedure is not acceptable for normal commercial drug production.

It has further been found that the prior art synthesis summarised earlier under the heading “Aminoboronate Procedure” results, when applied to the synthesis of TRI 50c or an ester thereof, in formation of an impurity designated Impurity IV:

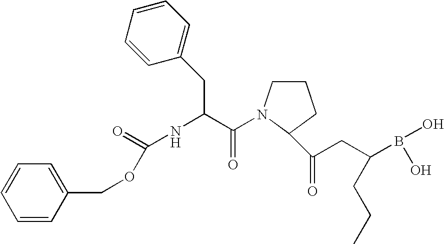

Attempts to separate Impurity IV from TRI 50c have not succeeded. The same applies to TRI 50c salts and esters and the corresponding salts and esters of Impurity IV. No purification technique which has been tried can prevent the presence of Impurity IV if said prior art synthesis is used.

Amongst other things, the synthetic methods described in this section of the specification addresses the problems of controlling C—B bond cleavage in organoboronic compounds as well as providing chirally purified salts of TRI 50c and other organoboronic acids on a commercial scale. In this regard, it has been found that C—B bonds seem to be cleaved by a non-oxidative mechanism which occurs in the presence of many solvents, including water and e.g. aqueous acids and bases, amongst others.

Chirally-selective precipitation may be used to recover organoboronic acids in high purity.

Thus C—B bond cleavage (and hence in particular generation of Impurity I) may be controlled by:
Selection of acetonitrile as a solvent, where a solvent is required in processing and acetonitrile has the necessary salvation power; in particular acetonitrile is selected in process where a polar solvent is desirable or necessary.
Avoiding excessive contact with water.

In terms of TRI 50c salt production, therefore, the disclosure includes processes comprising one, two or three of the following features:
(i) resolution of the (R,S,S) and (R,S,R) epimers of TRI 50c by chirally selective precipitation using diethanolamine and conveniently, but not necessarily, using as starting material TRI 50c in the form of an ester, for example the pinacol ester;
(ii) control of the duration and/or conditions of hydrolysis of TRI 50c diethanolamine ester, for example as obtained by such precipitation, to control C—B bond breakage;
(iii) use of acetonitrile as solvent for TRI 50c, for example as obtained by such hydrolysis, for the purposes of reacting the TRI 50c with a base to form the salt. Another favourable solvent can be tetrahydrofuran.

As an optional, or even stand-alone, fourth feature, TRI 50c salts may be dried by azeodrying using acetonitrile.

It is considered that C—B bond cleavage may occur by a nucleophilic mechanism, and the disclosure therefore includes methods in which opportunities for nucleophilic attack are minimised.

The above four features, or any one, two or three of them, may be applied to the manufacture and processing of other boronic compounds, particularly acids of formula (I) and their derivatives (e.g. esters and salts).

The disclosure provides in one aspect, therefore, the use of diethanolamine to resolve by selective precipitation the diastereomers of boronic acids of formula (I). The starting material may be an acid (I) or a derivative thereof capable of forming a diethanolamine ester of the boronic acid. The precipitation selects acids having a chiral centre C* of (R) configuration as precipitate. The precipitate may be recovered and converted to the corresponding boronic acid or a salt thereof. The salt may be made into a pharmaceutical formulation.

For optimised chiral purity and yield, the diethanolamine may be used in an amount of about 1.25±0.1 equivalents based on initial equivalents of boronic acid having a chiral centre C* of (R) configuration.

The initial boronic acid or acid derivative may for example comprise from 50% to 60% molecules having chiral centre C* of (R)-configuration and from 40% to 50% molecules having chiral centre C* of (S)-configuration.

The method opens the way to commercialisation of the boronic acids (I) and their derivatives, particularly salts, as pharmaceuticals. Commercial scale products and activities using the boronic acids (I) and their derivatives are therefore provided.

In one embodiment, there is provided a process for separating diastereomers of a boronic acid of formula (I), comprising:

combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration, and mixing to form a mixture;

causing or allowing the boronic species and the diethanolamine to react until a precipitate forms; and

recovering the precipitate.

When the starting material is an ester, it may be an ester of the boronic acid with an alcohol selected from the group consisting of alcohols whose sole potential electron donor heteroatoms are oxygens which, in the boronic ester, correspond to the oxygens of the ester functional group.

In some methods, the diethanolamine is in an amount of from 1.2 to 1.3 equivalents based on the boronic species in which chiral centre C* is of (R) configuration.

There are included processes in which the boronate species is an ester of the boronic acid and a diol, in particular a diol which is not sterically hindered. As exemplary diols may be mentioned pinacol, neopentylglycol, 1,2-ethanediol, 1,2-propanediol, 1,3-propanediol, 2,3-butanediol, 1,2-diisopropylethanediol, or 5,6-decanediol. A particular diol is pinacol.

The boronic species and the diethanolamine may be caused to react by heating the mixture to an elevated temperature, for example the mixture may be refluxed. e.g. for at least 10 hours.

The precipitate may be recovered by filtration. The recovered precipitate may be washed with diethylether. The recovered precipitate, after washing if such takes places, may be dissolved in a solvent selected from CH₂Cl₂ and CHCl₃ and reprecipitated by combining the resulting solution with diethylether. A particular solvent is CH₂Cl₂.

The recovered precipitate may be converted to the acid of formula (II), suitably by hydrolysis, for example by dissolving the precipitate in an organic solvent selected from e.g. halohydrocarbons and combinations thereof, agitating the resulting solution with an aqueous liquid, e.g. an aqueous acid having a pH of below 3, whereby the dissolved precipitate is converted to the formula (II) acid, and recovering the formula (II) acid by evaporation. The organic solvent may be CH₂Cl₂ or CHCl₃. A particular solvent is CH₂Cl₂. In some processes, organic solvent is further evaporated from the recovered formula (I) acid.

The disclosure includes methods in which an ester of a boronic acid (II), particularly a diethanolamine ester, is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes.

Thus the recovered precipitate referred to in the last paragraph but one may be hydrolysed using an aqueous acid, particularly 2% hydrochloric acid or another mineral acid of similar pH, for no more than about 30 minutes at about room temperature, or less. Suitably, the precipitate is dissolved in a non-nucleophilic organic solvent (e.g. a halohydrocarbon or halohydrocarbon mixture for example CH₂Cl₂) and the resulting solution is contacted with the aqueous acid for a period as previously described. The precipitate is thereby hydrolysed to form the free acid of formula (II), which remains in the organic solvent. The organic solvent may be separated from the aqueous medium and then evaporated to obtain solid acid of formula I.

There are included processes in which a formula (II) acid, for example obtained as described in the preceding paragraph, is dried. In a class of processes, the formula (II) acid is dried when it is in the organic solvent by contacting the solvent with a hygroscopic solid.

Included are processes in which the formula (II) acid, when in the organic solvent, is washed with an aqueous ammonium salt.

Chirally purified boronic acid may be converted to a pharmaceutically acceptable base addition salt thereof, in particular by dissolving the acid in acetonitrile, combining the resultant solution with an aqueous solution or suspension of a pharmaceutically acceptable base, and causing or allowing the base and the acid to react, then evaporating to dryness to obtain an evaporation residue. The step of causing or allowing the acid and the base to react may comprise agitating the combination of the acetonitrile solution of the acid and the aqueous solution or suspension of the base at a temperature of not more than 35° C. and often of not more than 30° C., e.g. not more than 25° C.; an optimal temperature is room temperature, in which case a reaction time of about 2 hours might be appropriate. The process may further comprise:
(i) redissolving the evaporation residue in acetonitrile and evaporating the resulting solution to dryness; and
(ii) repeating step (i) as often as necessary to obtain a dry evaporation residue.

In some processes the dry evaporation residue is dissolved in acetonitrile or tetrahydrofuran to form a solution, and the solution is combined with (e.g. slowly added to, at a rate sufficiently slow to avoid lump formation) a 3:1 to 1:3 v/v mixture of diethylether and an aliphatic or cycloaliphatic solvent to form a precipitate, said solution being added to the diethylether/(cyclo)aliphatic solvent mixture in a ratio (solution:mixture) of from 1:5 to 1:15 v/v. The precipitate is recovered and some or substantially all remaining solvent is removed from the recovered precipitate whilst maintaining the temperature at no more than 35° C., e.g. is removed under reduced pressure. Included are processes in which the temperature at the start of the drying process is about 10° C. and is increased during the process to 35° C. The aliphatic or cycloaliphatic solvent may have 6, 7 or 8 carbon atoms; the solvent may be an alkane, for example an n-alkane, e.g. n-heptane. Some reactions may be carried out at ambient temperature, which may e.g. be 15-30° C., e.g. 20-30° C.; sometimes ambient temperature may be room temperature.

The salts produced by the invention may contain a trace amount of the aliphatic or cycloaliphatic solvent, e.g. an amount of less than 0.1%, particularly less than 0.01%, for example an amount of about 0.005%.

In the process for making the salt, the base may comprise a cation of valency n and be used in a stoichiometry (boronic acid:base) of about n:1. In particular processes, the base is an alkali metal or alkaline earth metal base, for example an alkali metal hydroxide or an alkaline earth metal hydroxide. As one base may be mentioned sodium hydroxide. As another base may be mentioned calcium hydroxide. The disclosure includes processes in which the base is sodium hydroxide and the dry evaporation residue is dissolved in acetonitrile. The disclosure includes processes in which the base is calcium hydroxide and the dry evaporation residue is dissolved in tetrahydrofuran.

The disclosure is not limited as to the method by which the boronic acids of Formula (II) are obtained (for example as an ester thereof). However, in one class of subject matter, the Formula (II) acid has an R¹ group of the formula —(CH₂)ₛ—O—R³ in which R³ is methyl or ethyl and s is independently 2, 3 or 4, and the Formula (II) acid is prepared via an intermediate of Formula (XXV):

(HO)₂B—(CH₂)ₛ—O—R³ (XXV),

which intermediate is made by reaction between a borate ester and a suitable 1-metalloalkoxyalkane.

A novel aspect of the disclosure comprises the Formula (XXV) intermediates.

The Formula (XXV) intermediates may be made by reacting a 1-metalloalkoxyalkane, where the alkoxyalkane is of the formula —(CH₂)ₛ—O—R³, with a borate ester to form a compound of Formula (XXV).

It will be appreciated that the above method provides a general procedure for making alkoxyalkylboronic acids, which may be presented by the formula R^{Z}—O—R^{y}—B(OH)₂. Such alkoxyalkylboronic acids may be converted to aminoboronates, and the aminoboronates may be derivatised at their amino group to form an amide bond linked to another moiety. In other words, the aminoboronates may be converted to boropeptides. The method will now be described further with non-limiting reference to compounds of Formula (XXV).

The starting materials for the reaction may be a metalloalkoxyalkane, e.g. a Grignard reagent, obtainable from 1-haloalkoxyalkane of the formula Hal-(CH₂)ₛ—O—R³ (where Hal is a halogen) and a borate ester. The metal is in particular magnesium. Another metal is lithium, in which case the metallo reagent may be prepared by reacting the 1-haloalkoxyalkane with butyl lithium. Where the method includes preparation of the metallo reagent from the haloalkoxyalkane, the haloalkoxyalkane may be a chloroalkoxyalkane; the corresponding bromo compounds may also be used. To make a Grignard reagent, magnesium may be reacted with the haloalkoxyalkane.

Suitable borate esters are esters of mono- and di-functional alcohols (e.g. of EtOH, MeOH, BuOH, pinacol, glycol, pinanediol etc). For example, the ester may be of the formula B(OR^{a})(OR^{b})(OR^{c}) where R^{a}, R^{b} and R^{c} and C₁-C₄ alkyl and may be the same as each other.

An exemplary procedure for making a Formula (XXV) intermediate, illustrated with reference to methoxypropane as the alkoxyalkane species, is:

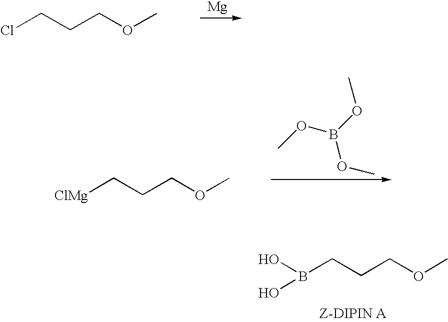

The reactions are suitably carried out in an organic solvent, e.g. THF.

The above-described procedure for making alkoxyalkylboronic acids avoids generation of Impurity IV (see above), or its analogues in those cases where the end product is not TRI 50c or a derivative (salt, ester etc) thereof. The procedure therefore provides a unique route to making TRI 50c, its esters and salts, uncontaminated by Impurity IV, and for making other aminoboronic acids which are substituted α- to the boron by an alkoxyalkyl group and are uncontaminated by impurities analogous to Impurity IV.

An alkoxyalkylboronic acid, i.e. a compound which may be represented by the formula R^{Z}—O—R^{Y}—B(OH)₂, may be converted to an aminoboronic compound, for example a boropeptide, by any suitable procedure, e.g. one known in the art. A reaction scheme for making alkoxyalkylboronic acids into aminoboronates, and for converting aminoboronates into peptide boronates is illustrated with reference to synthesis of TRI 50c at the start of the Examples of this specification. The reaction scheme may be modified as desired, e.g.: diethanolamine precipitation and subsequent steps may be omitted, and/or reagent substitutions may be made. For example, pinacol may be replaced by another diol. LDA is a non-nucleophilic strong base and may be replaced by another such base. Other examples include, but are not limited to, lithium diisopropylamide, lithium 2,2,6,6-tetramethylpiperidine, 1-lithium 4-methylpiperazide, 1,4-dilithium piperazide, lithium bis(trimethylsilyl) amide, sodium bis(trimethylsilyl)amide, potassium bis(trimethylsilyl)amide, isopropyl magnesium chloride, phenyl magnesium chloride, lithium diethylamide, and potassium tert-butoxide. The reactions may be carried out in any suitable solvent: where n-heptane is used in the Examples, it may be replaced by another inert non-polar solvent, e.g. another aliphatic or cycloaliphatic solvent, for example an alkane, e.g. an n-alkane.

It will be appreciated from the aforegoing that the above described methods may be used in the manufacture of organoboronic acids salts as described. It is not necessary for sequential steps to be carried out as one operation or at the same site: they may be performed in this way or different processes (different parts of the overall synthesis) may be distributed in time and/or space. Particular end product salts are monosodium, monolithium, hemicalcium and hemimagnesium salts, for example of TRI 50c.

Generally, the reactions may suitably be carried out with a non-nucleophilic solvent. Where a nucleophilic solvent is present, minimum contact is preferred, for example in the case of hydrolysis of diethanolamine esters.

### 6. High Purity Products

The “high purity products” of the disclosure include inter alia boronic acids, diethanolamine esters and salts obtainable by (having the characteristics of a product obtained by) the disclosed methods. Also included are products obtained directly or indirectly by the disclosed methods.

Particular products of the invention are base addition salts of a boronic acid of Formula (II) having the chiral purity of such salt when prepared by a method described herein. Other products are base addition salts of a boronic acid of Formula (II) having the purity of such salt when prepared by a method described herein.

Product identities will be apparent from the preceding description and the following examples. In addition, products of the disclosure are described in the claims. Of particular note are the data in Example 38, indicating that the processes of the invention can remarkably achieve end product salts free of impurities detectable by HPLC. In other instances, the salts are substantially free of impurities, e.g. at least 98% pure, more usually at least 99% pure, e.g. at least 99.5% pure, in terms of reverse phase (RP) HPLC percentage peak area. Salts may at least 99.3%, 99.4%, 99.5% 99.6%, 99.7%, 99.8% or 99.9% pure, in terms of reverse phase (RP) HPLC percentage peak area. Suitable RP HPLC procedures comply with reference 1 and/or reference 2 and/or reference 3 of Example 38. Included also are products at least substantially free of Impurity I and analogues, products free of Impurity IV and analogues, and products containing small traces of non-polar solvent, e.g. n-heptane. The trace amount of non-polar solvent may be less than 0.2%, 0.1%, 0.05%, 0.01% or 0.005% as determined by GC-headspace chromatography.

Included also are salts containing less than 410 ppm acetonitrile.

Some salts contain impurities of less than 10,000 ppm, 5000 ppm, 1000 ppm, or 500 ppm.

### Use of the Products of the Disclosure

The compounds of the disclosure are thrombin inhibitors. They are therefore useful for inhibiting thrombin. There are therefore provided compounds which have potential for controlling haemostasis and especially for inhibiting coagulation, for example in the treatment or prevention of secondary events after myocardial infarction. The medical use of the compounds may be prophylactic (including to treat thrombosis as well as to prevent occurrence of thrombosis) as well as therapeutic (including to prevent re-occurrence of thrombosis or secondary thrombotic events).

The compounds may be employed when an anti-thrombogenic agent is needed. Further, it has been found that the compounds, including those of boronic acids of Formula (III), are beneficial in that the class is useful for treating arterial thrombosis by therapy or prophylaxis. The disclosed compounds are thus indicated in the treatment or prophylaxis of thrombosis and hypercoagulability in blood and tissues of animals including man. The term “thrombosis” includes inter alia atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

In one method, the disclosed products are used to prevent thrombosis in surgery. In particular, the compounds of the present invention are used to prevent thrombosis during CABG surgery. Thus the disclosure contemplates medicaments to prevent thrombosis in the extracorporeal circuit during CABG surgery. As previously described, the compounds disclosed herein may also be used during CABG without the use of a cardiopulmonary bypass machine.

In particular, the compounds described herein are useful for the prevention of thrombosis in procedures involving an extracorporeal blood circuit, for example a surgical procedure, for example Coronary Artery Bypass Graft (CABG) surgery. The compounds of this disclosure may be incorporated into a cardiopulmonary bypass machine or may be administered externally to the extracorporeal blood circuit. More usually, they may be administered intravenously to the patient by infusion.

The products of the invention may be administered intravenously or through the extracorporeal blood flow, or by any other means which allows a rapid onset of action. The administration of the compounds is ideally controlled so that levels of therapeutic effect may be raised or lowered, as the situation requires.

It is known that hypercoagulability may lead to thromboembolic diseases. Examples of venous thromboembolism which may be treated or prevented with compounds of the disclosure include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the disclosure are useful for maintaining patency of indwelling catheters.

Examples of cardiogenic thromboembolism which may be treated or prevented with compounds of the disclosure include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of conditions involving arterial thrombosis include unstable angina (severe constrictive pain in chest of coronary origin), myocardial infarction (heart muscle cell death resulting from insufficient blood supply), ischemic heart disease (local ischemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterio-venous (mixed) thrombosis, anti-thrombotic compounds of the disclosure are useful for maintaining patency in arteriovenous shunts.

Other conditions associated with hypercoagulability and thromboembolic diseases which may be mentioned inherited or acquired deficiencies in heparin cofactor II, circulating antiphospholipid antibodies (Lupus anticoagulant), homocysteinemia, heparin induced thrombocytopenia and defects in fibrinolysis.

The thrombin inhibitors of the disclosure are thus indicated both in the therapeutic and/or prophylactic treatment of all the aforesaid disorders.

In one method, the products of the disclosure are used for the treatment of patients by haemodialysis, by providing the product in the dialysis solution, as described in relation to other thrombin inhibitors in WO 00/41715. The disclosure therefore includes dialysing solutions and dialysing concentrates which comprise a product of the disclosure, as well as a method of treatment by dialysis of a patient in need of such treatment, which method comprises the use of a dialysing solution including a low molecular weight thrombin inhibitor. Also included is the use of an anti-thrombotic product of the disclosure for the manufacture of a medicament for the treatment by dialysis of a patient, in which the anti-thrombotic product of the disclosure is provided in the dialysing solution.

In another method, the products of the disclosure are used to combat undesirable cell proliferation, as described in relation to other thrombin inhibitors in WO 01/41796. The undesirable cell proliferation is typically undesirable hyperplastic cell proliferation, for example proliferation of smooth muscle cells, especially vascular smooth muscle cells. The products of the disclosure particularly find application in the treatment of intimal hyperplasia one component of which is proliferation of smooth muscle cells. Restenosis can be considered to be due to neointimal hyperplasia; accordingly intimal hyperplasia in the context of the disclosure includes restenosis.

The products of the disclosure are also contemplated for the treatment of ischemic disorders. More particularly, they may be used in the treatment (whether therapeutic or prophylactic) of an ischemic disorder in a patient having, or at risk of, non-valvular atrial fibrillation (NVAF) as described in relation to other thrombin inhibitors in WO 02/36157. Ischemic disorders are conditions whose results include a restriction in blood flow to a part of the body. The term will be understood to include thrombosis and hypercoagulability in blood, tissues and/or organs. Particular uses that may be mentioned include the prevention and/or treatment of ischemic heart disease, myocardial infarction, systemic embolic events in e.g. the kidneys or spleen, and more particularly of cerebral ischemia, including cerebral thrombosis, cerebral embolism and/or cerebral ischemia associated with non-cerebral thrombosis or embolism (in other words the treatment (whether therapeutic or prophylactic) of thrombotic or ischemic stroke and of transient ischemic attack), particularly in patients with, or at risk of, NVAF.

The products of the disclosure are also contemplated for the treatment of rheumatic/arthritic disorders, as described in relation to other thrombin inhibitors in WO 03/007984. Thus, the products of the disclosure may be used in the treatment of chronic arthritis, rheumatoid arthritis, osteoarthritis or ankylosing spondylitis

Moreover, the products of the disclosure are expected to have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general. Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

The products of the disclosure are further indicated in the treatment of conditions where there is an undesirable excess of thrombin without signs of hypercoagulability, for example in neurodegenerative diseases such as Alzheimer's disease. In addition to its effects on the coagulation process, thrombin is known to activate a large number of cells (such as neutrophils, fibroblasts, endothelial cells and smooth muscle cells). Therefore, the compounds of the disclosure may also be useful for the therapeutic and/or prophylactic treatment of idiopathic and adult respiratory distress syndrome, pulmonary fibrosis following treatment with radiation or chemotherapy, septic shock, septicaemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

The compounds may also be useful in the treatment of pancreatitis.

The compounds described herein are further considered to be useful for inhibiting platelet procoagulant activity. The disclosure provides a method for inhibiting platelet pro-coagulant activity by administering a boronic acid compound described herein to a mammal at risk of, or suffering from, arterial thrombosis, particularly a human patient. Also provided is the use of such compounds for the manufacture of medicaments for inhibiting platelet procoagulant activity.

The use of products of the disclosure as inhibitors of platelet pro-coagulant activity is predicated on the observation that the boronic acids described herein are indicated to be effective at inhibiting arterial thrombosis as well as venous thrombosis.

Indications involving arterial thrombosis include acute coronary syndromes (especially myocardial infarction and unstable angina), cerebrovascular thrombosis and peripheral arterial occlusion and arterial thrombosis occurring as a result of atrial fibrillation, valvular heart disease, arterio-venous shunts, indwelling catheters or coronary stents. Accordingly, in another aspect there is provided a method of treating a disease or condition selected from this group of indications, comprising administering to a mammal, especially a human patient, a compound of the disclosure. The disclosure includes products for use in an arterial environment, e.g. a coronary stent or other arterial implant, having a coating which comprises a compound according to the disclosure.

The compounds of the disclosure may be used prophylactically to treat an individual believed to be at risk of suffering from arterial thrombosis or a condition or disease involving arterial thrombosis or therapeutically (including to prevent re-occurrence of thrombosis or secondary thrombotic events).

There is therefore included the use of selective thrombin inhibitors (e.g. base addition salts of organoboronic acids) described herein for treatment of the above disorders by prophylaxis or therapy as well as their use in pharmaceutical formulations and the manufacture of pharmaceutical formulations.

### Administration and Pharmaceutical Formulations

The compounds may be administered to a host, for example, in the case where the drug has anti-thrombogenic activity, to obtain an anti-thrombogenic effect. In the case of larger animals, such as humans, the compounds may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The compounds described herein may be administered intravenously by infusion for the purpose of preventing thrombosis during surgery. They may therefore be administered peri-operatively by infusion.

The compounds of the disclosure may be combined and/or co-administered with any cardiovascular treatment agent. There are large numbers of cardiovascular treatment agents available in commercial use, in clinical evaluation and in pre-clinical development, which could be selected for use with a product of the disclosure for the prevention of cardiovascular disorders by combination drug therapy. Such agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. abciximab, eptifibatide, tirofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic and an ACE (angiotensin converting enzyme) inhibitor.

The compounds of the disclosure may be combined and/or co-administered with any antithrombotic agent with a different mechanism of action, such as the antiplatelet agents acetylsalicylic acid, ticlopidine, clopidogrel, thromboxane receptor and/or synthetase inhibitors, prostacyclin mimetics and phosphodiesterase inhibitors and ADP-receptor (P₂ T) antagonists.

The products of the disclosure may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The compounds of the disclosure may be combined and/or co-administered with a cardioprotectant, for example an adenosine A1 or A3 receptor agonist.

There is also provided a method for treating an inflammatory disease in a patient that comprises treating the patient with a product of the disclosure and an NSAID, e.g., a COX-2 inhibitor. Such diseases include but are not limited to nephritis, systemic lupus, erythematosus, rheumatoid arthritis, glomerulonephritis, vasculitis and sarcoidosis. Accordingly, the anti-thrombotic compounds of the disclosure may be combined and/or co-administered with an NSAID.

Actual dosage levels of active ingredients in the pharmaceutical compositions of this disclosure may be varied so as to obtain an amount of the active compound(s) that is effective to achieve the desired therapeutic response for a particular patient, composition, and mode of administration (referred to herein as a “therapeutically effective amount”). The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

According to a further aspect there is provided a parenteral formulation including a compound as described herein. The formulation may consist of the compound alone or it may contain additional components, in particular the compound may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration.

The formulations and dosage forms of the disclosure include those in which the active agent is a base addition salt, in particular those in which the salt is an alkali metal salt, for example a lithium, sodium or potassium salt, of which sodium salts may be mentioned as particular salts. Another class of formulations contains aminosugar salts of the disclosed boronic acids, for example N-methyl-D-glucamine salts. The salts mentioned in this paragraph may be administered as solutions in water, typically containing one or more additives, for example isotonicity agent(s) and/or antioxidant(s). A suitable way to store the salts is in solid form, for example as dry powder, and to make them up into solutions for administration prior to administration.

It will be understood from the aforegoing that there are provided pharmaceutical products comprising a mono alkali metal or hemi alkaline earth metal salt of a boronic acid of Formula (II) in the form of a product suitable parenteral formulation. The salt may be a lyophilisate.

It is currently contemplated that, in the case of parenteral administration, for example i.v. administration, of TRI 50c or derivatives thereof (e.g. base addition salts), the active compound (e.g. salt) might for instance be administered in an amount of from 0.5 to 2.5 mg/Kg e.g. over a maximum period of 72 hours, calculated as TRI 50c. Other acids, in whatever form (free acid, salt or prodrug) might be administered in equivalent molar amounts. The disclosure is not limited to administration in such quantities or regimens and includes dosages and regimens outside those described in the previous sentence.

In the case of cardiovascular or cardiac surgery, for example coronary artery bypass grafting (with or without cardiopulmonary bypass) or valve repair or replacement, the patient suffers a large thrombogenic stimulus and relatively high levels of anticoagulation are required. The amount of anticoagulation can be measured by the activated clotting time (ACT). In experiments conducted using TRI 50c monosodium salt in the dog, it was found that the ACT should be >300 seconds before surgical intervention commences. The activated clotting time (ACT) is a commonly used parameter for assessing the degree of anticoagulation during cardiac surgery. It has been found suitable in settings such as these for administration of the antithrombotic boronic acid salt to commence with a bolus administration of the compound, e.g. 25 mg of TRI 50c monosodium salt over one minute, followed by infusion at a rate of about 100-300 mg of TRI 50c monosodium salt per hour, e.g. up to about 160 mg/hour. It should be noted for the case of administration of another salt, e.g. the calcium salt, that these rates of administration may readily be calculated, using the conversion factor referred to previously, as equivalent to approximately a bolus administration of about 0.044 mmoles TRI 50c followed by infusion at a rate of about 0.18-0.53 mmoles TRI 50c per hour, e.g. up to about 0.28 mmoles per hour. Administration at a rate of about 0.18-0.53 millimoles TRI 50c per hour is therefore contemplated to be suitable for other TRI 50c compounds (e.g. salts) and for other boronic acids of similar potency (e.g. administered as base addition salts) in the setting of CPB or cardiac surgery, e.g. CABG with or without CPB. Of course, the rate of administration may be adjusted according to clinical judgement, for example to take account of a patient's weight or other factors, and a rate of administration of about 0.1-0.75 mmoles/hour may therefore be mentioned. In the case of dissimilar potencies, say (Ki values outside the range of 5-25 nM), dosage rate may be adjusted accordingly. The disclosure is not limited to administration in such quantities or regimens and includes dosages and regimens outside those described in this paragraph.

In instances where there is a lower thrombogenic stimulus, it is contemplated that intravenous TR 50c monososium salt would be administered to adult patients (in fact, normally into the extracorporeal blood stream) at a lesser rate, for example of no more than, say, 50 mg/hour, e.g. from about 20 to about 50 mg/hour, equivalent to approximately 0.035-0.089 millimoles TRI 50c per hour. Administration at a rate of up to about 0.089 millimoles TRI 50c per hour, e.g. about 0.035-0.089 millimoles TRI 50c per hour, is therefore contemplated to be suitable for other TRI 50c salts and for salts of other boronic acids of similar potency (Ki of TRI 50c=7-22 nM) in the setting of CIHD and other intermittent apheresis procedures. Of course, the rate of infusion may be adjusted in the clinical judgement of a medical practitioner, for example to take account of a patient's weight or other factors, and a rate of infusion of up to about 0.125 mmoles/hour, e.g. about 0.025-0.125 mmoles/hour may therefore be mentioned. In the case of dissimilar potencies, say (Ki values outside the range of 5-25 nM), dosage rate may be adjusted accordingly. In order to avoid adding excessive water to the blood in CIHD, a relatively concentrated solution is desirably infused, for example it is contemplated that a concentration of at least 35 mmolar is preferable (in terms of concentration of the active boronyl species, e.g. TRI 50c and its corresponding boronate ions), although lower concentrations of, say, 18 mmolar cannot be altogether excluded. The disclosure is not limited to administration in such quantities or regimens and includes dosages and regimens outside those described in this paragraph.

It is generally desirable in instances of renal failure or disorder that as little water as possible be added during anticoagulation. It is therefore contemplated that a relatively soluble antithrombotic will be used, e.g. a sodium salt or a reaction product of a boronic acid and an aminosugar (for example N-methyl-D-glucamine) in the case of CIHD. Other procedures which involve intravenous anticoagulation may be less sensitive to the volume of water injected or infused and less soluble products may be preferred on a balance of factors. Thus, for example, it may in such instances be preferred to administer a salt of a divalent metal such as calcium or zinc for reason of stability. Magnesium is another pharmaceutically acceptable divalent metal. Trivalent metals may also be mentioned.

Examples of the procedures or settings less sensitive to volume of added water referred to in the previous paragraph include:
Surgery, for example cardiovascular or cardiac surgery (e.g. CABG with or without CPB), surgery involving CPB, orthopaedic surgery such as total hip replacement, total knee replacement, major hip or knee surgery; general surgery on patients at high risk of thrombosis, such as abdominal or pelvic surgery for cancer;
Prevention of venous thromboembolic events (e.g. deep vein thrombosis and/or pulmonary embolism). Examples include patients who have suffered or are suspected of having suffered a thrombotic event; and patients bedridden for more than 3 days and with acute cardiac failure, acute respiratory failure, infection
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples)
Other acute treatments used in relation to indications indicated previously, except in the setting of renal failure or disorder.

Preferably, the salts are capable of having a rapid onset and a short duration of action, thus allowing the patients' platelet coagulation levels to return to a safe level quickly, e.g. 30 mins after termination of administration.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration. The preparation may also be administered directly into the extracorporeal blood circuit during, for example CABG surgery.

Liquid dosage forms for parenteral administration may include solutions, suspensions, liposome formulations, or emulsions in oily or aqueous vehicles. In addition to the active compounds, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

It has been found that the base addition salts of the disclosure are more soluble if they are dissolved into an aqueous liquid which is free of acid than if they are dissolved under pH-controlled condition to keep the pH at or close to 7. In particular, it has been found possible to form surprisingly concentrated solutions (of up to about 600 mg/ml in the case of TRI 50c monosodium salt) at a pH of about 9.5 if the pH is not controlled, in contrast to a control solution where a maximum concentration of about 20 mg/ml is obtained at about pH 7.

Solid dosage forms for parenteral administration may encompass solid and semi-solid forms and may include pellets, powders, granules, patches, and gels. In such solid dosage forms, the active compound is typically mixed with at least one inert, pharmaceutically acceptable excipient or carrier.

The disclosed salts may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The parenteral formulations may be prepared as large volume parenterals (LVPs), e.g. larger than 100 ml, more particularly about 250 ml, of a liquid formulation of the active compound. Examples of LVPs are infusion bags. The parenteral formulations may alternatively be prepared as small volume parenterals (SVPs), e.g. about 100 ml or less of a liquid formulation of the active compound. Examples of SVPs are vials with solution, vials for reconstitution, prefilled syringes for injection and dual chamber syringe devices.

The formulations of the disclosure include those in which the salt is an alkali metal salt, for example a lithium, sodium or potassium salt, of which sodium salts may be mentioned as particular salts. Another class of formulations contains aminosugar salts of the disclosed boronic acids, for example N-methyl-D-glucamine salts. The salts mentioned in this paragraph may be administered as solutions in water, typically containing one or more additives, for example isotonicity agent(s) and/or antioxidant(s). A suitable way to store the salts is in solid form, for example as dry powder, and to make them up into solutions for administration prior to administration.

One class of formulations disclosed herein is intravenous formulations. For intravenously administered formulations, the active compound or compounds can be present at varying concentrations, with a carrier acceptable for parenteral preparations making up the remainder. Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a salt of disclosure in dissolved form, the solvent may have dissolved therein one or more other substances, for example an antioxidant and/or an isotonicity agent. As organic cosolvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form. As previously indicated, the disclosure includes formulations of alkali metal salts of the disclosed boronic acids, e.g. TRI 50c, having a solvent which consists of water.

The solubility of the active compound in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would be administered at a pH of between 6.0 and 7.0 such as a pH of 6.5.

The parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active compound or compounds with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed salts might not be stored in solution but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

One class of formulations disclosed herein comprises subcutaneous or intradermal formulations (for example formulations for injection) in which the active salt (or active agent combination) is formulated into a parenteral preparation that can be injected subcutaneously or intradermally. The formulation for administration will comprise the active salt and a liquid carrier.

The carrier utilized in a parenteral preparation that will be injected subcutaneously or intradermally may be an aqueous carrier (for example water, typically containing an additive e.g. an antioxidant and/or an isotonicity agent) or a nonaqueous carrier (again one or more additives may be incorporated). As a non-aqueous carrier for such parenteral preparations may be mentioned highly purified olive oil.

The active compound and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed salts will not be stored in liquid formulation but as dry solids, in order to prolong shelf life.

For making subcutaneous implants, the active salt may suitably be formulated together with one or more polymers that are gradually eroded or degraded when in use, e.g. silicone polymers, ethylene vinylacetate, polyethylene or polypropylene.

Transdermal formulations may be prepared in the form of matrices or membranes, or as fluid or viscous formulations in oil or hydrogels or as a compressed powder pellet. For transdermal patches, an adhesive which is compatible with the skin may be included, such as polyacrylate, a silicone adhesive or polyisobutylene, as well as a foil made of, e.g., polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Transdermal formulations may be prepared according to a method disclosed in, e.g., B W Barry, “Dermatological Formulations, Percutaneous Absorption”, Marcel Dekker Inc., New York—Basel, 1983, or Y W Chien, “Transdermal Controlled Systemic Medications”, Marcel Dekker Inc., New York—Basel, 1987.

It will be understood from the aforegoing that there are provided pharmaceutical products comprising an alkali metal salt, particularly sodium salt, of a boronic acid of Formula (II) in dry fine particle form, suitable for reconstitution into an aqueous read-to-use parenteral formulation. The alkali metal salt is suitably an acid salt. The alkali metal salt may be in a small volume parenteral unit dosage form. The alkali metal salt may be presented in a form, e.g. dry powder form, suitable for reconstituting as a large volume parenteral. One example is a sodium salt of a boronic acid of Formula (II), particularly TRI 50c, in dry powder form for reconstitution as a liquid intravenous formulation (solution) containing a tonicity agent, particularly sodium chloride. The dry powder form of a salt used in a parenteral formulation may be a lyophilisate. The reconstituted solution may be administered by injection or infusion.

### EXAMPLES

### Examples 1 to 4

### Introductory Remarks

### Apparatus

Throughout the following procedures of Examples 1 to 4, standard laboratory glassware and, where appropriate, specialised apparatus for handling and transferring of air sensitive reagents are used.

All glassware is heated at 140-160° C. for at least 4 hours before use and then cooled either in a desiccator or by assembling hot and purging with a stream of dry nitrogen.

### Solvents

The organic solvents used in the procedures of Examples 1 to 4 are all dry. Suitably, they are dried over sodium wire before use.

### Dryness

In the drying procedures of Example 1 to 4, products are tested for dryness (including dryness in terms of organic solvent) by observing weight loss on drying. The following procedure was followed to determine loss on drying: a sample was placed in a vacuum drier and dried at 40° C. at 100 mbar for 2 hours. Products are considered dry when the decrease in weight upon drying is less than 0.5% of the total weight of the starting material.

Examples 1 to 4 describe performance of the following reaction scheme and conversion of the resultant TRI 50c to sodium and calcium salts thereof:

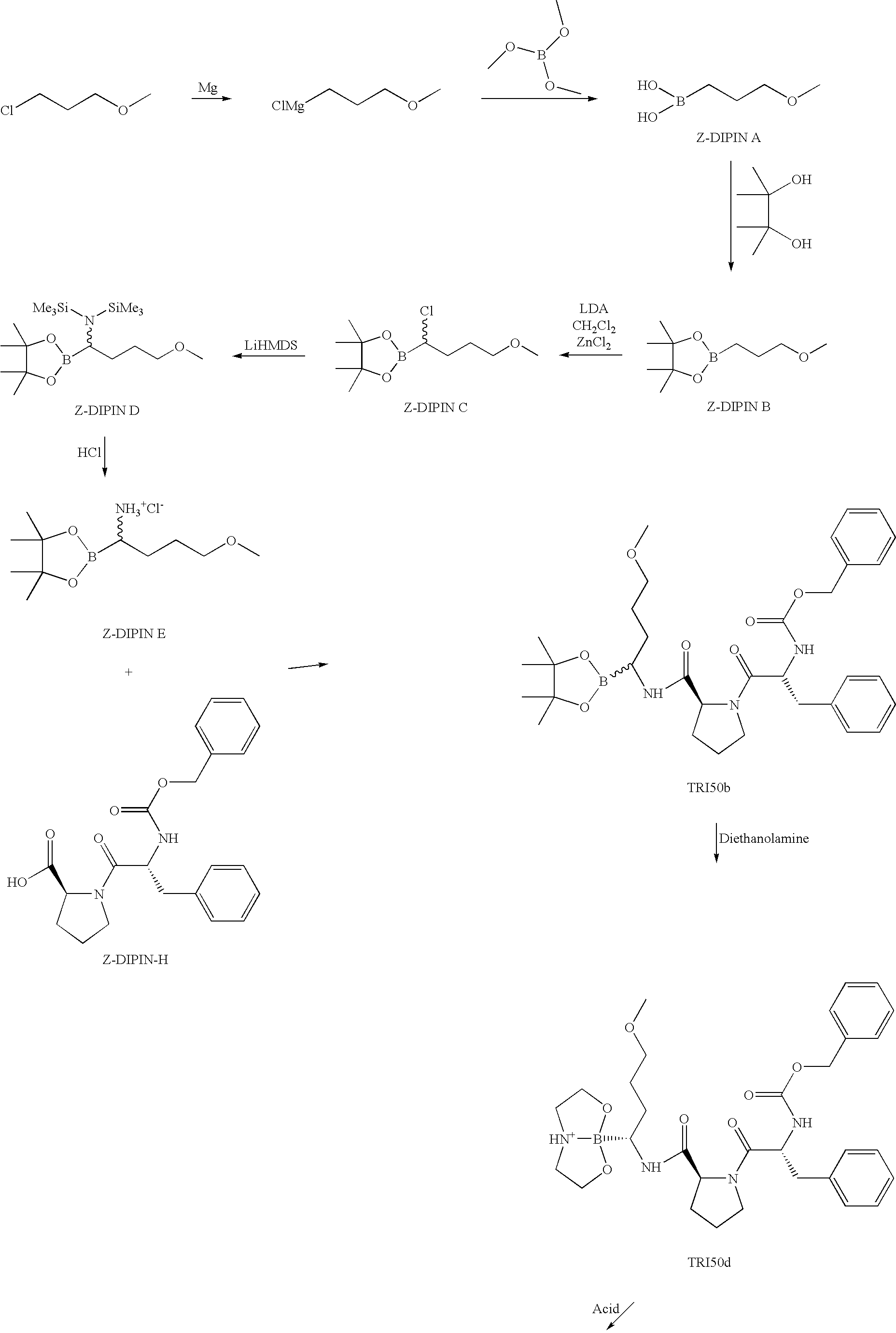

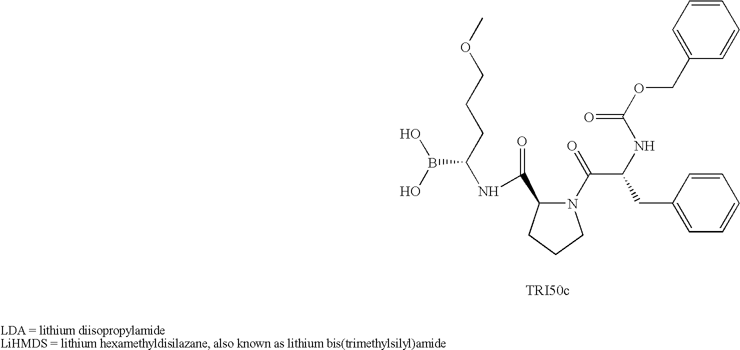

### Example 1

### Synthesis of TRI 50B

### Step 1: Z-DIPIN B

### Procedure A

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux until all the magnesium is consumed. After the reaction is finished, the reaction mixture is cooled to ambient temperature and slowly added to a solution of 64.4 g (620 mmole) trimethylborate in 95 ml dry tetrahydrofuran; the latter solution is cooled to below 0° C. and, if it warms up during the course of the reaction, the reaction mixture must be added to it sufficiently slowly to maintain the temperature of this solution below 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The layers are allowed to settle and the phases are separated. The aqueous layer is rewashed three times with 200 ml tert.-butylmethylether. The combined organic layers are allowed to settle and additional water separated from this solution is removed. The organic layer is dried over magnesium sulfate, filtered and evaporated to dryness. The evaporation residue is filtered from the precipitated solid and the filtrate dissolved in 175 ml toluene. 34.8 g (292 mmole) pinacol is charged to the solution followed by stirring at ambient temperature for not less than 10 hours. The solution is evaporated to dryness, dissolved in 475 ml n-heptane and washed three times with 290 ml saturated aqueous solution of sodium hydrogen carbonate. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70%) Z-DIPIN B (oil)

### Procedure B

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux. After the reaction is finished, the reaction mixture is cooled to ambient temperature and slowly added to a solution of 64.4 g (620 mmole) trimethylborate in 95 ml dry tetrahydrofuran, maintaining the temperature of this solution below minus 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The organic solvent is removed by distillation under vacuum. 300 ml n-heptane is charged to the aqueous solution of the evaporation residue followed by addition of 34.8 g (292 mmole) pinacol. The two-phase-mixture is stirred at ambient temperature for not less than 2 hours. After allowing the layers to settle, the aqueous phase is separated. 300 ml n-heptane is charged to the aqueous solution and the two-phase-mixture is stirred at ambient temperature for not less than 2 hours. After allowing the layers to settle, the aqueous phase is separated. The organic layers are combined and washed once with 200 ml water, followed by 200 ml saturated sodium hydrogen carbonate solution and two further washes with 200 ml water each. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70-85%) Z-DIPIN B (oil)

### Step 2: Z-DIPIN C

16.6 g (164 mmole) diisopropylamine and 220 ml tetrahydrofuran are charged and cooled to −30 to −40° C. To this solution 41.8 g (163 mmole) n-butyl lithium, 25% in n-heptane is added, followed by stirring at 0 to −5° C. for one hour. This freshly prepared solution of lithium diisopropylamide is cooled to −30° C. and then added to a solution of 27.9 g (139 mmole) Z-DIPIN B in 120 ml tetrahydrofuran and 35.5 g (418 mmole) dichloromethane at a temperature between −60 and −75° C. The solution is stirred at that temperature for half an hour followed by addition of 480 ml (240 mmole) 0.5N anhydrous Zinc(II)-chloride in tetrahydrofuran or 32.5 g (240 mmole) anhydrous solid Zinc(II)-chloride. After stirring at −65° C. for one hour, the reaction mixture is allowed to warm to ambient temperature and stirred for another 16-18 hours. The reaction mixture is evaporated to dryness (i.e. until solvent is removed) and followed by addition of 385 ml n-heptane. The reaction mixture is washed with 150 ml 5% sulfuric acid, with 190 ml saturated sodium hydrogen carbonate solution, and 180 ml saturated sodium chloride solution. The organic layer is dried over magnesium sulfate, filtered and evaporated to dryness (i.e. until solvent is removed). The oily residue is transferred into the next step without further purification.

Yield: 19 g (55%) Z-DIPIN C

### Step 3: Z-DIPIN D

To a solution of 23.8 g (148 mmole) hexamethyldisilazane in 400 ml tetrahydrofuran at −15° C. is added 34.7 g (136 mmole) n-butyl lithium, 25% in n-heptane and stirred for one hour. The solution is cooled to −55° C. followed by the addition of 30.6 g (123 mmole) Z-DIPIN C dissolved in 290 ml tetrahydrofuran and 35 ml tetrahydrofuran to this freshly prepared solution of LiHMDS. The solution is allowed to warm to ambient temperature and stirred for 12 hours. The reaction mixture is evaporated to dryness, the evaporation residue dissolved in 174 ml n-heptane, washed with 170 ml water and 75 ml saturated sodium chloride solution. The organic phase is dried over magnesium sulfate, filtered and evaporated to complete dryness (i.e. until solvent is removed). The oily residue is dissolved in 100 g n-heptane. This solution is carried over into the next step without further purification.

Yield: 32.2 g (70%) Z-DIPIN D

### Step 4: Z-DIPIN (TRI50b, Crude)

A solution of 26.6 g (71 mmole) Z-DIPIN D in 82.6 g n-heptane is diluted with 60 ml n-heptane and cooled to −60° C. followed by introduction of 10.5 g (285 mmole) hydrogen chloride. The reaction mixture is subsequently evacuated and flushed with nitrogen, while the temperature is increased in increments of about 20° C. to ambient temperature. The solvent is removed from the oily precipitate and replaced several times by 60 ml fresh n-heptane. The oily residue is dissolved in 60 ml tetrahydrofuran (Solution A).

To a different flask 130 ml tetrahydrofuran, 24.5 g (61.5 mmole) Z-D-Phe-Pro-OH and 6.22 g (61.5 mmole) N-methylmorpholine are charged and cooled to −20° C. To this solution a solution of 8.4 g (61.5 mmole) isobutylchloroformate in 20 ml tetrahydrofuran is added and stirred for 30 minutes, followed by addition of Solution A at −25° C. Upon complete addition, up to 16 ml (115 mmole) triethylamine is added to adjust the pH to 9-10, measured using a pH stick. The reaction mixture is allowed to warm to ambient temperature and stirred for 3 hours, still under nitrogen. The solvent is evaporated to dryness and the evaporation residue dissolved in 340 ml tert.-butylmethylether (t-BME). The solution of Z-DIPIN in t-BME is washed twice with 175 ml 1.5% hydrochloric acid. The combined acidic washes are given a rewash with 175 ml t-BME. The combined organic layers are washed with 175 ml water, with 175 ml saturated sodium hydrogen carbonate solution, with 175 ml 25% sodium chloride solution, dried over magnesium sulfate and filtered. This solution is carried over into the next step without further purification.

Yield: 29.9 g (80%) Z-DIPIN

### Example 2

### Synthesis of TRI 50D

### Diethanolamine Adduct of TRI 50C

The starting material used in this Example is the solution of TRI 50b (“Z-DIPIN”) obtained in Example 1. The solution is carried forward to the synthesis of TRI 50d without further purification. The solution of Z-DIPIN in t-BME (containing 7.0 g (11.5 mmole) (R,S,R) TRI50b, calculated based on HPLC results of Z-DIPIN) is evaporated to dryness and the evaporation residue dissolved in 80 ml diethylether. 1.51 g (14.4 mmole) diethanolamine is added and the mixture heated at reflux for at least 10 hours, during which process the product precipitates. The suspension is cooled to 5-10° C., filtered and the filter residue washed with diethylether.

To improve chiral and chemical purity the wet filter cake (7 g) is dissolved in 7 ml dichloromethane, cooled to 0-5° C. and the product precipitated by addition of 42 ml diethylether and filtered. The isolated wet product is dried at 35° C. in vacuum or at least 4 hours, until day.

Yield: 5.5 g (80%) Tri50d

Melting Point: 140-145° C.

### Example 3

### Preparation of Sodium Salt of TRI50C

1.5 kg (2.5 mole) TRI50d from Example 2 is dissolved in 10.5 L dichloromethane. 11 L 2% hydrochloric acid is added and the mixture is stirred for at most 30 minutes (optimally about 20 minutes) at room temperature. A precipitate forms in the organic phase. After stirring, the layers are allowed to settle and separated. The aqueous layer is rewashed twice with 2.2 L dichloromethane. The combined organic layers are washed with a solution of 625 g ammonium chloride in 2.25 L water. (The ammonium chloride buffers the pH of the aqueous extractions to be within a range of from about pH 1-2 to about pH 4-5, as strongly acidic conditions might cleave peptide bonds). The organic phase is dried over magnesium sulfate, filtered and the filtrate evaporated to dryness. An assay of the free boronic acid is performed (by the RP HPLC method of Example 38 for at most 30 mins (optionally about 20 min) at room temperature) and the amounts of the solvents and base for conversion of the acid to the salt are calculated. If 2.5 mol of the free acid is obtained, the evaporation residue is dissolved in 5 L acetonitrile followed by addition of a solution of 100 g (2.5 mole) sodium hydroxide as a 5% solution in 2.2 L water. The solution is stirred for two hours at ambient temperature (e.g. 15-30° C., optimally room temperature) and then evaporated in vacuum (of ca. 10 mmHg) at a temperature not exceeding 35° C. The evaporation residue is repeatedly dissolved in 3.5 L fresh acetonitrile and evaporated to dryness to remove traces of water. If the evaporation residue is dry, it is dissolved in 3 L acetonitrile (or alternatively in 6 L THF) and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum at a temperature initially of about 10° C. and then increasing to a limit of about 35° C., until dry.

Yield: 1.0 kg (70%) Tri50c sodium salt.

### Example 4

### Preparation of Calcium Salt of TRI50C

1.5 kg (2.5 mole) TRI50d from Example 2 is dissolved in 10.5 L dichloromethane. 11 L 2% hydrochloric acid is added and the mixture is stirred for at most 30 minutes (optimally about 20 minutes) at room temperature. After stirring the layers are allowed to settle and separated. The aqueous layer is given a rewashed twice with 2.2 L dichloromethane. The combined organic layers are washed with a solution of 625 g ammonium chloride in 2.25 L water. The organic phase is dried over magnesium sulfate, filtered and the filtrate evaporated to dryness. An assay of the free boronic acid is performed and the amounts of the solvents and base for conversion of the acid to the salt are calculated. If 2.5 mol of the free acid is obtained, the evaporation residue is dissolved in 5 L acetonitrile followed by addition of a suspension of 93 g (1.25 mole) calcium hydroxide in 1 L water. The solution is stirred for two hours at ambient temperature (e.g. 15-30° C., optimally room temperature) and then evaporated under vacuum (of ca. 10 mmHg) at a temperature initially of about 10° C. and then increasing to a limit of about 35° C. The evaporation residue is repeatedly dissolved in 3.5 L fresh acetonitrile and evaporated to dryness to remove traces of water. If the evaporation residue is dry, it is dissolved in 6 L-tetrahydrofuran and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum (of ca. 10 mmHg) at a temperature below 35° C. until dry.

Yield: 0.98 kg (70%) Tri50c calcium salt.

The procedures of Examples 1 to 4 may be scaled up and, if operated carefully, will produce highly pure salts. In the diethanolamine precipitation step it is important to use 1.25 equivalents of diethanolamine per equivalent of (R,S,R) TRI 50b. In the hydrolysis of the diethanolamine ester, it is important to avoid excessively long contact with the aqueous acid. Likewise the TRI 50b should be synthesised via the Grignard reaction to Z-DIPIN A.

### Example 5

### Alternative Conversion of TRI 50B to TRI 50C

The synthetic procedures described in this and subsequent synthetic examples were generally performed under nitrogen and using dry solvents as supplied from commercial sources.

1. Approximately 300 g of TRI 50b, obtained by the HPLC purification of racemic TRI 50b) were dissolved in approximately 2.5 L diethylether. It is estimated that different batches of TRI 50b had isomeric purities ranging from 85% R,S,R to in excess of 95% R,S,R.

2. Approximately 54 ml diethanolamine were added (1:1 stoichiometry with total TRI 50b content), and the mixture was refluxed at 40° C.

3. The precipitated product was removed, washed several times with diethylether and dried.

4. The dry product was dissolved in CHCl₃. Hydrochloric acid (pH 1) was added and the mixture was stirred approximately 1 h at room temperature.

5. The organic layer was removed and washed with NH₄Cl solution.

6. The organic solvent was distilled off and the residual solid product was dried.

Typical yield: Approximately 230 g

### Example 6

### Preparation of Lithium Salt of TRI5c

Cbz-Phe-Pro-BoroMpg-OH obtained by the method of Example 5 (20.00 g, 38.1 mM) is dissolved in acetonitrile (200 ml) with stirring at room temperature. To this solution is added LiOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water necessary with light warming for about 20 minutes. The solution is filtered through filter paper and evacuated to dryness, again with the temperature of the solution not exceeding 37° C. The resultant product is dried under vacuum overnight to normally yield a white brittle solid.

The salt was then dried under vacuum over silica to constant weight (72 h).

Yield 17.89 g.

Microanalysis:

| C % Found | H % Found | N % Found | B % Found | Metal % Found |
|---|---|---|---|---|
| (Calc.) | (Calc.) | (Calc.) | (Calc.) | (Calc.) |
| 57.14 | 6.60 | 7.34 | 2.07 | Li 1.26 |
| (61.03) | (6.64) | (7.90) | (2.03) | (1.31) |

### Example 7

### UV/Visible Spectra of Lithium Salt of TRI50C

UV/Visible spectra of the salt resulting from the procedure of Example 6 were recorded in distilled water at 20° C. from 190 nm to 400 nm. The salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 451

### Example 8

### Aqueous Solubility of Lithium Salt of TRI50C

The salt used in this Example was made using a modification of the process described in Example 6. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material. The lithium salt was comparatively soluble and so was redissolved at 50 mg/ml in the same manner previously described.

Solubility when dissolved at 25 mg/ml: 43 mM (23 mg/ml).

Solubility when dissolved at 50 mg/ml: 81 mM (43 mg/ml).

### Example 9

### Preparation of Sodium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added NaOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 15-20 minutes. The solution is filtered through filter paper and evacuated to dryness, again with the temperature of the solution not exceeding 37° C. The resultant product is dried under vacuum overnight to normally yield a white brittle solid. The product may be present as an oil or tacky solid due to residual water, in which case it is dissolved in ethyl acetate and evacuated to dryness to produce the product as a white solid.

The salt was then dried under vacuum over silica to constant weight (72 h).

Yield: Over 50%.

Microanalysis:

| C % Found | H % Found | N % Found | B % Found | Metal % Found |
|---|---|---|---|---|
| (Calc.) | (Calc.) | (Calc.) | (Calc.) | (Calc.) |
| 59.93 | 6.47 | 7.31 | 1.91 | Na 3.81 |
| (59.24) | (6.44) | (7.67) | (1.98) | (4.20) |

### Example 10

### UV/Visible Spectra of Sodium Salt of TRI50C

UV/Visible spectra of the sodium salt resulting from the procedure of Example 9 were recorded in distilled water at 20° C. from 190 nm to 400 nm. The salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 415.

### Example 11

### Aqueous Solubility of Sodium Salt of TRI50C

The salt used in this Example was made using a modification of the process described in Example 9. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material. The sodium salt was comparatively soluble and so was redissolved at 50 mg/ml in the same manner previously described.

Solubility when dissolved at 25 mg/ml: 44 mM (25 mg/ml).

Solubility when dissolved at 50 mg/ml: 90 mM (50 mg/ml).

### Example 12

### Preparation of Potassium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added KOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 1 L distilled water with warming to 37° C. for about 2 hours. The solution is filtered through filter paper and evacuated to dryness, again with the temperature of the solution not exceeding 37° C. The resultant product is dried under vacuum overnight to normally yield a white brittle solid.

Yield: 14.45 mg.

The salt was then dried under vacuum over silica to constant weight (72 h).

Microanalysis:

| C % Found | H % Found | N % Found | B % Found | Metal % Found |
|---|---|---|---|---|
| (Calc.) | (Calc.) | (Calc.) | (Calc.) | (Calc.) |
| 54.84 | 6.25 | 7.02 | 2.01 | K 4.29 |
| (57.55) | (6.26) | (7.45) | (1.92) | (6.94) |

### Example 13

### UV/Visible Spectra of Potassium Salt of TRI50C

UV/Visible spectra of the potassium salt resulting from the procedure of Example 12 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 438.

### Example 14

### Aqueous Solubility of Potassium Salt of TRI50C

The salt used in this Example was made using a modification of the process described in Example 12. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 29 mM (16 mg/ml).

### Example 15

### Preparation of Zinc Salt of TRI 50C

The relative solubility of zinc hydroxide is such that, if the hydroxide had been used to prepare the corresponding TRI 50c salt using the procedure of Example 6, they would not have resulted in homogeneous salt formation. A new method was therefore developed to prepare the zinc salt, as described in this and the next examples.

TRI 50c sodium salt (2.24 g, 4.10 mM) was dissolved in distilled water (100 ml) at room temperature and zinc chloride in THF (4.27 ml, 0.5M) was carefully added with stirring. A white precipitate that immediately formed was filtered off and washed with distilled water. This solid was dissolved in ethyl acetate and washed with distilled water (2×50 ml). The organic solution was evacuated to dryness and the white solid produced dried over silica in a desiccator for 3 days before microanalysis. Yield 1.20 g.

¹H NMR 400 MHz, δ_{H} (CD₃OD) 7.23-7.33 (20H, m, ArH), 5.14 (4H, m, PhCH₂O), 4.52 (4H, m, aCH), 3.65 (2H, m), 3.31 (12H, m), 3.23 (6H, s, OCH₃), 2.96 (4H, d, J=7.8 Hz), 2.78 (2H, m), 2.58 (2H, m), 1.86 (6H, m), 1.40 (10H, m).

¹³C NMR 75 MHz δ_{C} (CD₃OD) 178.50, 159.00, 138.05, 137.66, 130.54, 129.62, 129.50, 129.07, 128.79, 128.22, 73.90, 67.90, 58.64, 58.18, 56.02, 38.81, 30.06, 28.57, 28.36, 25.29.

FRIR (KBr disc) νₘₐₓ (cm⁻¹) 3291.1, 3062.7, 3031.1, 2932.9, 2875.7, 2346.0, 1956.2, 1711.8, 1647.6, 1536.0, 1498.2, 1452.1, 1392.4, 1343.1, 1253.8, 1116.8, 1084.3, 1027.7, 916.0, 887.6, 748.6, 699.4, 595.5, 506.5.

### Example 16

### Preparation of Arginine Salt of TRI5C

Cbz-Phe-Pro-BoroMpg-OH obtained by the method of Example 5 (20.00 g, 38.1 mM) is dissolved in acetonitrile (200 ml) with stirring at room temperature. To this solution is added arginine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 2 L distilled water with warming to 37° C. for 2 hours. The solution is filtered through filter paper and evacuated to dryness, again with the temperature of the solution not exceeding 37° C. The resultant product is dried under vacuum overnight to normally yield a white brittle solid.

The salt was then dried under vacuum over silica to constant weight (72 h).

Yield: 10.54 g.

Microanalysis:

| C % Found | H % Found | N % Found | B % Found |
|---|---|---|---|
| (Calc.) | (Calc.) | (Calc.) | (Calc.) |
| 52.47 | 7.12 | 15.25 | 1.52 |
| (56.65) | (7.20) | (14.01) | (1.54) |

### Example 17

### UV/Visible Spectra of Arginine Salt of TRI50C

UV/Visible spectra of the salt resulting from the procedure of Example 15 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 406.

### Example 18

### Aqueous Solubility of Arginine Salt of TRI50C

The salt used in this Example was made using a modification of the process described in Example 16. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 14 mM (10 mg/ml).

### Example 19

### Preparation of Lysine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added L-lysine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 3 L distilled water with warming to 37° C. for 2 hours. The solution is filtered through filter paper and evacuated to dryness, again with the temperature of the solution not exceeding 37° C. The resultant product is dried under vacuum overnight to normally yield a white brittle solid. The product may be present as an oil or tacky solid (due to residual water), in which case it is then dissolved in ethyl acetate and evacuated to dryness to produce the product as a white solid.

The salt was then dried under vacuum over silica to constant weight (72 h).

Yield: 17.89.

Microanalysis:

| C % Found | H % Found | N % Found | B % Found |
|---|---|---|---|
| (Calc.) | (Calc.) | (Calc.) | (Calc.) |
| 57.03 | 7.43 | 10.50 | 1.72 |
| (59.11) | (7.36) | (10.44) | (1.61) |

### Example 20

### UV/Visible Spectra of Lysine Salt of TRI50C

UV/Visible spectra of the salt resulting from the procedure of Example 19 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 437.

### Example 21

### Aqueous Solubility Of Lysine Salt of TRI50C

The salt used in this Example was made using a modification of the process described in Example 19. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 13 mM (8.6 mg/ml).

### Example 22

### Preparation of N-Methyl-D-Glucamine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added N-methyl-D-glucamine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 20 minutes. The solution is filtered through filer paper and evacuated to dryness, again with the temperature of the solution not exceeding 37° C., or freeze dried. The resultant product is dried under vacuum overnight to normally yield a white brittle solid.

The salt was then dried under vacuum over silica to constant weight (72 h).

Yield: 21.31 g.

Microanalysis:

| C % Found | H % Found | N % Found | B % Found |
|---|---|---|---|
| (Calc.) | (Calc.) | (Calc.) | (Calc.) |
| 56.67 | 7.28 | 7.74 | 1.63 |
| (56.67) | (7.41) | (7.77) | (1.50) |

### Example 23

### UV/Visible Spectra of N-Methyl-D-Glucamine Salt of TRI50C

UV/Visible spectra of the salt resulting from the procedure of Example 22 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 433.

### Example 24

### Aqueous Solubility of N-Methyl-D-Glucamine Salt of TRI50C

The salt used in this Example was made using a modification of the process described in Example 22. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt was observed to fully dissolve. The salt was comparatively soluble and so was redissolved at 50 mg/ml in the same manner previously described.

Solubility when dissolved at 25 mg/ml: 35 mM (25 mg/ml).

Solubility when dissolved at 50 mg/ml: 70 mM (50 mg/ml).

### Example 25

### Alternative Preparation of Arginine Salt of TRI50C

The arginine salt is formed simply by adding a slight molar excess of L-arginine to a solution of 0.2-0.3 mmol of TRI50c in 10 ml of ethyl acetate. The solvent is evaporated after one hour, and the residue is triturated twice with hexane to remove excess arginine.

### Example 26

### First Preparation of Calcium Salt of TRI 50C

Cbz-Phe-Pro-BoroMpg-OH (20.00 g, 38.1 mM) obtained by the method of Example 5 is dissolved in acetonitrile (200 ml) with stirring at room temperature. To this solution is added Ca(OH)₂ as a 0.1M solution in distilled water (190 ml). The resultant clear solution is stirred for 2 hours at room temperature and then evacuated to dryness under vacuum with its temperature not exceeding 37° C. The resultant product is a white brittle solid.

The salt was then dried under vacuum over silica to constant weight (72 h).

Yield: 17.69 g.

### Example 27

### Second Alternative Preparation of Calcium Salt of TRI 50C

50.0 g TRI 50c (95.2 mmol) were dissolved under stirring in 250 ml acetonitrile at room temperature and then cooled with an ice bath. To this ice cooled solution 100 ml of an aqueous suspension of 3.5 g (47.6 mmol) calcium hydroxide was added dropwise, stirred for 2.5 hours at room temperature, filtered and the resulting mixture evaporated to dryness, the temperature not exceeding 35° C. The clear yellowish oily residue was redissolved in 200 ml acetone and evaporated to dryness. The procedure of redissolving in acetone was repeated one more time to obtain colourless foam.

This foam was redissolved in 100 ml acetone, filtered and added dropwise to an ice cooled solution of 1100 ml petrol ether 40/60 and 1100 ml diethylether. The resulting colourless precipitate was filtered, washed two times with petrol ether 40/60 and dried under high vacuum, yielding 49.48 g of a colourless solid (92%), with a purity of 99.4% according to an HPLC measurement.

### Example 28

### UV/Visible Spectra of Calcium Salt of TRI 50C

UV/Visible spectra of the salt resulting from the procedure of Example 26 were recorded in distilled water at 20° C. from 190 nm to 400 nm. TRI 50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
l the path length of the UV cell
and ε is the extinction coefficient.

Extinction coefficient: 955.

### Example 29

### Aqueous Solubility of Calcium Salt of TRI 50C

The salt used in this Example was made using a modification of the process described in Example 27. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 5 mM (5 mg/ml).

### Example 30

### In Vitro Activity of Calcium Salt of TRI 50C

TRI 50c calcium salt was assayed as an inhibitor of human α-thrombin by an amidolytic assay (J. Deadman et al, J. Med. Chem. 38:15111-1522, 1995, which reports a Ki value of 7 nM for TRI 50b).

The inhibition of human α-thrombin therefore, was determined by the inhibition of the enzyme catalysed hydrolysis of three different concentrations of the chromogenic substrate S-2238.

200 μl of sample or buffer and 50 μl of S-2238 were incubated at 37° C. for 1 minute and 50 μl of human α-thrombin (0.25 NIHμ/ml) was added. The initial rate of inhibited and uninhibited reactions were recorded at 4.5 nm. The increase in optical density was plotted according to the method of Lineweaver and Burke. The Km and apparent Km were determined and Ki was calculated using the relationship.

$$V = \frac{V max}{1 + \frac{Km}{[ S ]} \cdot ( 1 + \frac{[ I ]}{Ki} )}$$

The buffer used contained 0.1M sodium phosphate, 0.2M NaCl, 0.5% PEG and 0.02% sodium azide, adjusted to pH 7.5 with orthophosphoric acid.

The samples consist of the compound dissolved in DMSO.

The reader is referred to Dixon, M. and Webb, E. C., “Enzymes”, third edition, 1979, Academic Press, the disclosure of which is incorporated herein by reference, for a further description of the measurement of Ki.

TRI 50c calcium salt was observed to have a Ki of 10 nM.

### Example 31

### Preparation of Magnesium Salt of Tri 50C

TRI 50c (1.00 g, 1.90 mM) was dissolved in methanol (10 ml) and stirred at room temperature. To this solution was added magnesium methoxide (Mg(CH₃O)₂) in methanol (1.05 ml, 7.84 wt %). This solution was stirred for 2 hours at room temperature filtered and evacuated to 5 ml. Water (25 ml) was then added and the solution evacuated down to dryness to yield a white solid. This was dried over silica for 72 hours before being sent for microanalysis. Yield 760 mg.

¹H NMR 300 MHz, δ_{H} (CD₃C(O)CD₃) 7.14-7.22 (20H, m), 6.90 (2H, m), 4.89 (4H, m, PhCH₂O), 4.38 (2H, m), 3.40 (2H, br s), 2.73-3.17 (20H, broad unresolved multiplets), 1.05-2.10 (16H, broad unresolved multiplets).

¹³C NMR 75 MHz δ_{C} (CD₃C(O)CD₃) 206.56, 138.30, 130.76, 129.64, 129.31, 129.19, 129.09, 128.20, 128.04, 74.23, 73.55, 67.78, 58.76, 56.37, 56.03, 48.38, 47.87, 39.00, 25.42, 25.29.

FTIR (KBr disc) νₘₐₓ (cm⁻¹) 3331.3, 3031.4, 2935.3, 2876.9, 2341.9, 1956.1, 1711.6, 1639.9, 1534.3, 1498.1, 1453.0, 1255.3, 1115.3, 1084.6, 1027.6, 917.3, 748.9, 699.6, 594.9, 504.5, 467.8.

### Example 32

### Solubility of TRI50C

The UV/visible spectra of TRI 50c resulting from the procedure of Example 5 and its solubility were obtained as described above in relation to the salts. The solubility of TRI5c when dissolved at 50 mg/ml was 8 mM (4 mg/ml).

### Example 33

### Analysis of Sodium, Calcium, Magnesium and Zinc Salts of (R,S,R) TRI 50C

The following salts were prepared using a boronate:metal stoichiometry of n:1, where n is the valency of the metal, using (R,S,R) TRI 50c of higher chiral purity than that used to prepare the salts described in Examples 8, 11, 14, 18, 21, 24 and 29.

### A. Sodium Salt (Product of Example 9)

Analytical data

HPLC or LC/MS: HPLC betabasic C18 Column,
CH₃CN, Water
Estimated Purity: >95% by UV (λ₂₁₅ₙₘ)

Micro analysis:

| | | Calcd. | Found. |
|---|---|---|---|
| C: | | 59.24 | 59.93 |
| H: | | 6.44 | 6.47 |
| N: | | 7.67 | 7.31 |
| Other: | B: | 1.98 | 1.91 |
| | Na: | 4.20 | 3.81 |

Physical Properties

Form: Amorphous solid
Colour: White
Melting Point: N/A
Solubility: Soluble in aqueous media
ca~50 mg/ml
M_{w}: 547.40

### B. Calcium Salt (Product of Example 26)

Analytical data

HPLC or LC/MS: HPLC betabasic C18 Column,
CH₃CN, Water
Estimated Purity: >95% by UV (λ₂₁₅ₙₘ)

Micro analysis:

| | | Calcd. | Found. |
|---|---|---|---|
| C: | | 59.27 | 55.08 |
| H: | | 6.48 | 6.43 |
| N: | | 7.71 | 7.08 |
| Other: | B: | 1.99 | 2.01 |
| | Ca: | 3.68 | 3.65 |

Physical Properties

Form: Amorphous solid
Colour: White
Melting Point: N/A
Solubility: Soluble in aqueous media
ca~4 mg/ml
M_{w}: 1088.89

### C. Manesium Salt (Product of Example 31)

Analytical data

HPLC or LC/MS: HPLC betabasic C18 Column,
CH₃CN, Water
Estimated Purity: >90% by UV (λ₂₁₅ₙₘ)

Micro analysis:

| | | Calcd. | Found. |
|---|---|---|---|
| C: | | 60.44 | 57.25 |
| H: | | 6.57 | 6.71 |
| N: | | 7.83 | 7.45 |
| Other: | B: | 2.01 | 2.02 |
| | Mg: | 2.26 | 2.12 |

Physical Properties

Form: Amorphous solid
Colour: White
Melting Point: N/A
Solubility: Soluble in aqueous media
ca~7 mg/ml
M_{w}: 1073.12

### D. Zinc Salt (Product of Example 15)

Analytical data

HPLC or LC/MS: HPLC betabasic C18 Column,
CH₃CN, Water
Estimated Purity: >95% by UV (λ_{215 nm})

Micro analysis:

| | | Calcd. | Found. |
|---|---|---|---|
| C: | | 58.21 | 56.20 |
| H: | | 6.33 | 6.33 |
| N: | | 7.54 | 7.18 |
| Other: | B: | 1.94 | 1.84 |
| | Zn: | 5.87 | 7.26 |

Physical Properties

Form: Amorphous solid
Colour: White
Melting Point: N/A
Solubility: Soluble in aqueous media
ca~2 mg/ml
M_{w}: 1114.18

Notes: The trigonal formula of the acid boronate is used in the calculated microanalyses. It is believed that a lower sodium salt solubility is reported in example 11 because the salt tested in example 11 had lower chiral purity.

### Conclusion

The zinc, calcium and magnesium salts have all been prepared with a stoichiometry of one metal ion to two molecules of TRI 50c. The values found for the calcium and magnesium salts are close to and thus consistent with those calculated for this 1:2 stoichiometry. For the zinc salt an excess of zinc was found; nonetheless, the zinc salt comprises a significant proportion of acid boronate. The sodium salt has been prepared with a stoichiometry of one metal ion to one molecule of TRI 50c. The value found for the sodium salt is close to and thus consistent with that calculated for this 1:1 stoichiometry.

### Example 34

### Stability

An assay of TRI 50c and its sodium and lysine salts before and after drying.

### 1. Tabulated Results

TABLE 1

| Compound | Amount [μg/mL] | Purity (% area) |
|---|---|---|
| TRI 50c dry | 1000.0 | 82.00 |
| TRI 50c non-dried | 947.3 | 85.54 |
| TRI 50c Na salt dry | 1024 | 98.81 |
| TRI 50c Na salt non-dried | 1005.8 | 98.61 |
| TRI 50c Lys salt dry | 813.3 | 90.17 |
| TRI 50c Lys salt non-dried | 809.8 | 92.25 |

The purity of the acid was lowered by the drying process but the purity of the salts was less affected; the purity of the sodium salt was not significantly reduced. Large differences in response factors will reduce the actual impurity levels, however.

### 2. Analytical Procedure

### 2.1 Sample Preparation

TRI 50c and its Na, Li and Lys salts were weighed into HPLC vials and stored in a desiccator over phosphorus pentoxide for 1 week. For sample analysis, 5 mg of dried and non-dried material was weighed in a 5 mL volumetric flask and dissolved in 1 mL acetonitrile and filled up with demineralised water to 5 mL.

### 3. Data Evaluation

The quantitative evaluation was performed using an HPLC-PDA method.

### 4. Analytical Parameters

### 4.1 Equipment and Software

| | |
|---|---|
| Autosampler | Waters Alliance 2795 |
| Pump | Waters Alliance 2795 |
| Column oven | Waters Alliance 2795 |
| Detection | Waters 996 diode array, MS-ZQ 2000 single quad |
| Software version | Waters Millennium Release 4.0 |

### 4.2 Stationary Phase

| | |
|---|---|
| Analytical Column ID | S71 |
| Material | X-Terra ™ C₁₈, 5 μm |
| Supplier | Waters, Eschborn, Germany |
| Dimensions | 150 mm × 2.1 mm (length, internal diameter) |

### 4.3 Mobile Phase

Aqueous phase: A: H₂O+0.1%
Organic phase: C: ACN

### Gradient Conditions:

| Time | Flow | % A | % C |
|---|---|---|---|
| 0.00 | 0.5 | 90 | 10 |
| 27.0 | 0.5 | 10 | 90 |
| 27.1 | 0.5 | 90 | 10 |
| 30.0 | 0.5 | 90 | 10 |

This example indicates that the salts of the disclosure, particularly the metal salts, e.g. alkali metal salts, are more stable than the acids, notably TRI 50c.

### Example 35

### In Vitro Assay as Thrombin Inhibitor of Magnesium Salt of TRI 50C

### Thrombin Amidolytic Assay

TRI 50c magnesium salt (TRI 1405) was tested in a thrombin amidolytic assay.

### Reagents:

### Assay Buffer:

100 mM Na phosphate
200 mM NaCl (11.688 g/l)
0.5% PEG 6000 (5 g/l)
0.02% Na azide
pH 7.5

Chromogenic substrate S2238 dissolved to 4 mM (25 mg+10 ml) in water. Diluted to 50 uM with assay buffer for use in assay at 5 μM. (S2238 is H-D-Phe-Pip-Arg-pNA).

Thrombin obtained from HTI, via Cambridge Bioscience, and aliquoted at 1 mg/ml with assay buffer. Dilute to 100 ng/ml with assay buffer and then a further 1 in 3 for use in the assay.

### Assay:

110 μl assay buffer
50 ul 5 μg/ml thrombin
2 μl vehicle or compound solution

### 5 min at 37° C.

### 20 μl 50 μM S2238

Read at 405 nm at 37° C. for 10 minutes and record Vmax

### Results and Discussion:

In this assay the magnesium salt of TRI 50c was found to show the same activity as TRI 50b as an external control.

### Example 36

### Intravenous Administration of TRI 50C Sodium Salt

The pharmacokinetics (PK) and pharmacodynamics (PD) of TRI 50c sodium salt were studied in beagle dogs following intravenous administration.

The PD was measured as thrombin time and APTT using an automated coagulometer. Plasma concentrations were measured using an LCMS/MS method.

TRI 50c monosodium salt (108.8 g) was dissolved in 0.9% sodium chloride (100 ml) and dosed i.v. at 1.0 mg/kg (1.0 ml/kg over 30 seconds). Blood samples were taken into 3.8% tri-sodium citrate (1+8) at pre dose, 2, 5, 10, 20, 30, minutes post dose and then at 1, 2, 3, 4, 6, 8, 12 and 24 hours post dose. Plasma was prepared by centrifugation and frozen at minus 20° C. pending analysis.

### Results

The sodium salt was tolerated well with no adverse events for the total duration of the study.

Male and female dogs responded similarly with a pharmacodynamic C max: at 2 minutes (thrombin time of 154 seconds raised from a base line of 14.3 seconds). Thrombin time was 26 seconds at one hour post dose.

There was an exceptionally good therapeutic ratio between the APTT and thrombin clotting time in dogs receiving the sodium salt at a dose of 1.0 mg/kg i.v. Thrombin clotting time was elevated 10.8 times above base line (154.4 seconds from 14.3 seconds) two minutes following dosing, compared to only 1.3 times elevation in the APTT (19 seconds to 25 seconds post dose).

### Example 37

### Residual n-Heptane of TRI 50C Calcium Salt

Salt prepared following the methods of Examples 1 and 3 was tested by headspace gas chromatography. Data are shown below:

Residual solvents: Headspace gas chromatography

| | |
|---|---|
| GC Parameter: | |
| Column: | DB-wax, 30 m, 0.32 mm ID, 5μ |
| Carrier Gas: | Helium 5.0, 80 kPas |
| Detector: | FID, 220° C. |
| Injector Temp: | 150° C. |
| Operating Conditions: | 35° C./7 min; 10° C./min up to 80° C./2 min; |
| | 40° C. up to 180° C./2 min |
| Injection volume: | 1 ml |
| Split: | On |
| Headspace Parameter: | |
| Oven temperature: | 70° C. |
| Needle temperature: | 90° C. |
| Transfer temperature: | 100° C. |
| Other parameters: | temper time: 15 min, GC-cycle time: 28 min, |
| | injection time: 0.03 min, duration: 0.4 min |

Calibration Standards: sample weight/dilution

| | | | concentration | area (average, |
|---|---|---|---|---|
| standard | weight (mg) | volume (ml) | (mg/ml) | n = 3) |
| n-heptane | 103.12 | 100 | 1.0312 | 2757.74756 |

| | | | concentration |
|---|---|---|---|
| sample no. | weight (mg) | volume (ml) | (mg/ml) |
| 1 | 100.84 | 5 | 20.17 |
| 2 | 99.12 | 5 | 19.82 |
| 3 | 100.03 | 5 | 20.01 |

n-heptane

| sample | concentration (mg/ml) | content (%) |
|---|---|---|
| 1 | 0.0010 | 0.0048 |
| 2 | 0.0009 | 0.0044 |
| 3 | 0.0010 | 0.0050 |
| | 0.00095 | 0.005 |

### Example 38

### HPLC Chromatograms

TRI 50c monosodium salt made by the method of Examples 1, 2 & 3 and TRI 50c hemicalcium salt made by the method of Examples 1, 2 & 4 were analysed by HPLC chromatography.

### 1. Method

### 1.1 Equipment and Software

| | |
|---|---|
| Autosampler | Waters Alliance 2795 |
| Pump | Waters Alliance 2795 |
| Column oven | Waters Alliance 2795 |
| Detection | Waters 2996 diode array, MS-ZQ single |
| | quad |
| Software version | Waters Millennium 4.0 |

### 1.2 Stationary Phase

| | |
|---|---|
| Analytical Column ID | S-71 |
| Material | XTerra ™ MS C₁₈, 5 μm |
| Supplier | Waters, Eschborn, Germany |
| Dimension | 150 mm × 2.1 mm (length, ID) |
| Pre-column ID | no pre-column |

Xterra MS C₁₈, 5 μm is a column packing material supplied by Waters Corporation, 34 Maple Street, Milford, Mass. 01757, US and local offices, as in years 2002/2003. It comprises hybrid organic/inorganic particles, consisting of spherical particles of 5 μm size, 125 Å pore size and 15.5% carbon load.

### 1.3 Mobile Phase

Aqueous phase: A: H₂O+0.1% HCOOH
Organic phase: C: ACN
H₂O═H₂O by Ultra Clear water purification system
ACN=gradient grade acetonitrile

### Gradient Conditions

| time | | | flow | gradient |
|---|---|---|---|---|
| [min] | A % | C % | [mL/min] | shape |
| 0.0 | 90.0 | 10.0 | 0.5 | |
| 27.00 | 10.0 | 90.0 | 0.5 | linear |
| 27.10 | 90.0 | 10.0 | 0.5 | linear |
| 30.00 | 90.0 | 10.0 | 0.5 | linear |

### 1.4 Instrumental Parameters

| | |
|---|---|
| Flow | 0.5 mL-min⁻¹ |
| Temperature | 40 ± 5° C. |
| HPLC control | Waters Millennium Release 4.0 |
| Calculation | Waters Millenium 4.0 |

### 2. Parameters

2.1 Wavelength/Retention Time/Response factors

TABLE
retention and detection parameter (k′ F: 0.5 ml/min, t0 = 0.9 mL/min)

| | | | | | Reciprocal |
|---|---|---|---|---|---|
| | RetTime | λ | | response factor | Response |
| Substance | [min] | [nm] | m/z | [area/μg] | factor |
| TRI 50c | 11.68 | 258 | 508.33 | 660 | 1 |
| Benzyl alcohol | 3.862 | 258 | n.d. | 1960 | 0.337 |
| Benzaldehyde | 6.13 | 258 | n.d. | 79939 | 0.0083 |
| Benzoic acid | 5.52 | 258 | n.d. | 5967 | 0.111 |
| Impurity I | 11.18 | 258 | 396.17 | 886 | 0.745 |
| Impurity II | 13.39 | 258 | 482.22 | 552 | 1.196 |

### 2.2 Linearity

Linearity Range 4000-10 μg/mL (detection UV 258 nm)

TABLE
Linearity data UV 258 nm

| calibration | area | target conc. | conc. found¹ |
|---|---|---|---|
| solution | [μAU's] | [μg/mL] | [μg/mL] |
| TRI 50c | 5353 | 10 | 20.44 |
| TRI 50c | 5301 | 10 | 20.37 |
| TRI 50c | 65809 | 100 | 113.35 |
| TRI 50c | 66365 | 100 | 114.17 |
| TRI 50c | 172019 | 250 | 270.43 |
| TRI 50c | 162587 | 250 | 256.48 |
| TRI 50c | 339503 | 500 | 518.13 |
| TRI 50c | 326912 | 500 | 499.51 |
| TRI 50c | 659257 | 1000 | 991.02 |
| TRI 50c | 647495 | 1000 | 973.63 |
| TRI 50c | 1322371 | 2000 | 1971.72 |
| TRI 50c | 1305196 | 2000 | 1946.32 |
| TRI 50c | 2724410 | 4000 | 4045.24 |

¹recalculated with linear equation

### Linear Equation Parameters:

Y=6.75e+002 X−8.45e+003
r=0.99975
r²=0.99950
Linearity Range 10-0.10 μg/mL (detection SIR m/z 508,33)

TABLE
Linearity data SIR 508.33

| calibration | mean area | target conc. | conc. found¹ |
|---|---|---|---|
| solution | [μAU's] | [μg/mL] | [μg/mL] |
| TRI 50c | 2188860 | 0.01 | 0.022 |
| TRI 50c | 2702839 | 0.01 | 0.045 |
| TRI 50c | 3817226 | 0.1 | 0.094 |
| TRI 50c | 3833799 | 0.1 | 0.095 |
| TRI 50c | 23153550 | 1 | 0.947 |
| TRI 50c | 24646892 | 1 | 1.013 |
| TRI 50c | 223007852 | 10 | 9.765 |
| TRI 50c | 233753043 | 10 | 10.239 |

¹recalculated with linear equation

### Equation Parameter

Y 2.27e+007 X+1.69e+006
r=0.99958
r²=0.99916

### 2.3 Quantitation Limit

The quantitation limit was determined using the signal to noise ratio criterion S/N>19,

UV 258 nm: 10 μg/mL
M/z 508.3: 0.1 μg/mL

### 2.4 Precision

| | Target concentration | | Amount | Retention time |
|---|---|---|---|---|
| Injection | [μg/mL] | Area | [μg/mL] | [min] |
| 1 | 250 | 165805 | 261.24 | 11.690 |
| 2 | 250 | 168644 | 265.44 | 11.662 |
| 3 | 250 | 167858 | 264.27 | 11.686 |
| 4 | 250 | 166947 | 262.93 | 11.692 |
| 5 | 250 | 166925 | 262.89 | 11.679 |
| 6 | 250 | 166294 | 261.96 | 11.696 |
| Mean | | 167079 | 263.12 | 11.684 |
| Std. Dev. | | 1033 | 1.528 | 0.01 |
| % RSD | | 0.6 | 0.6 | 0.1 |

### 2.5 Robustness

TABLE
robustness data; Standard 250 μg/mL aqueous solution
(containing <1% ACN)

| calibration | temp./time | area | recovery |
|---|---|---|---|
| solution | [° C./h] | [μAU's] | [%] |
| 250 μg/mL Tri50c | — | 172020 | — |
| 250 μg/mL Tri50c | 4° C. 16 h | 166294 | 96.67 |
| 2.5 μg/mL TRI50c | — | 88034891 | — |
| 2.5 μg/mL TRI50c | 37° C. 4 h | 88833175 | 100.9 |

### REFERENCES

1. ICH HARMONISED TRIPARTITE GUIDELINE. TEXT ON VALIDATION OF ANALYTICAL PROCEDURES Recommended for Adoption at Step 4 of the ICH Process on 27 Oct. 1994 by the ICH Steering Committee
2. FDA Reviewer Guidance. Validation of chromatographic methods. Center for Drug Evaluation and Research. November 1994
3. USP 23. <621> Chromatography
4. L. Huber. Validation of analytical Methods. LC-GC International February 1998
5. Handbuch Validierung in der Analytik. Dr. Stavros Kromidas (Ed.) Wiley-VCH Verlag. 2000. ISBN 3-527-29811-8

### 3. Results

3.1 Sample Name: TRI 50c monosodium salt

Injection volume: 10 μL

| | | | | Peak |
|---|---|---|---|---|
| | Ret Time | | Area | Height |
| Name | (Min) | Area % | [μAU's] | μAU |
| TRI 50c | 12.136 | 100.0000 | 604.27228 | 32.05369 |

3.2 Sample Name: TRI 50c Hemicalcium Salt

Injection volume: 10 μL

| | | | | Peak |
|---|---|---|---|---|
| | Ret Time | | Area | Height |
| Name | (Min) | Area % | [μAU's] | μAU |
| TRI 50c | 12.126 | 100.0000 | 597.11279 | 32.29640 |

The disclosed methods have been used to obtain salts substantially free of C—B bond degradation products, in particular salts containing no such products in an amount detectable by HPLC, specifically the method of Example 38. The disclosed methods have been used to obtain salts substantially free of Impurity I, in particular containing no Impurity I in an amount detectable by HPLC, specifically by the method of Example 38. The disclosed methods have been used to obtain salts substantially free of Impurity IV, in particular containing no Impurity IV in an amount detectable by HPLC, specifically by the method of Example 38.

### Example 39

### Determination of Diastereomeric Excess

TRI 50b, crude, contains three chiral centres. Two of them are fixed by the use of enantiomerically pure amino acids ((R)-Phe and (S)-Pro). The third one is formed during the synthesis. The favoured epimer is the desired TRI 50b, Isomer I (R,S,R-TRI 50b). Both epimers of TRI 50b are clearly baseline separated by the HPLC method, thus allowing determination of the diasteromeric excess (de) of TRI 50b.

TRI 50d is not stable under the conditions applied for HPLC purity determination, but decomposes rapidly on sample preparation to TRI 50c, so that TRI 50d and TRI 50c show the same HPLC traces.

The two isomers of TRI 50c are not baseline separated in HPLC, but both isomers are clearly visible. This becomes obvious, when TRI 50b, crude (mixture of both isomers) is converted with phenylboronic acid to TRI 50c, crude. Both isomers of TRI 50c are observed in HPLC nearly at the same relation as before in TRI 50b, crude.

Upon synthesis of TRI 50d from TRI 50b, crude, only one diastereoisomer is precipitated. In this case HPLC shows only one peak for TRI 50c, where a very small fronting is observed. Precipitation from dichloromethane/diethylether removes the fronting efficiently. The level of removal of Isomer II cannot be quantified by this HPLC method. Therefore samples before reprecipitation and after one and two reprecipitations were esterified with pinacol and the resulting samples of TRI 50b analysed by HPLC. Thus a de of 95.4% was determined for the crude sample. The reprecipitated sample resulted in a de of 99.0% and finally the sample that was reprecipitated twice showed a de of 99.5%.

These results clearly show the preferred precipitation of Isomer I, whereas Isomer II remains in solution.

### Example 40

### Intravenous Administration into Humans

### Trial Protocol

TGN 255, the monosodium salt of TRI 50c, was administered intravenously to 18 healthy male subjects as a single intravenous dose (randomised double blind placebo study). The test consisted of three groups, each of six males. From each group 5 men were given the active ingredient and 1 was given a placebo.

Total I.V. administered doses were:
82 mg (7 mg intravenous bolus (over 30 s) followed by an infusion of 25 mg/h for 3 hours).
130 mg (10 mg intravenous bolus (over 30 s) followed by an infusion of 40 mg/h for 3 hours).
120 mg (by infusion of 40 mg/h for 3 hours).

### Trial Results

1) No clinically significant findings were detected in any safety assessments. There were no adverse clinical events of either a general or cardiovascular nature during the study period of 24 hours for any dose of TGN 255.
2) The disposition of TGN 255 followed a pattern with a short distribution phase occurring within 30 minutes after the end of infusion and was eliminated from the plasma with an overall terminal elimination half life of about 4 hours.
3) Intravenous infusion of TGN 255 induced a rapid, dose-related increase in the thrombin time (TT). Within 30 minutes after cessation of infusion there was a fall to a level at which clinically significant anticoagulation is not expected.

### Example 41

### Mitral Valve Repair

Intravenous direct thrombin inhibitor TGN 255 (TRI 50c monosodium salt) was evaluated in dogs undergoing hypothermic CPB. Following a dose ranging study in conscious dogs, six beagle dogs were placed on CPB and underwent a simulated mitral valve repair. A range of dynamic coagulation markers were measured, including thrombin clotting time (TT), activated partial thromboplastin time (APTT), activated clotting time (ACT), ecarin clotting time (ECT), whole blood thrombin clotting time, platelet counts and function tests. In addition, pre and post-operative echocardiograms and intra-operative blood loss were evaluated. TGN 255 was shown to provide excellent anticoagulation during bypass and surgery with little bleeding. The short half life of TGN 255 was an advantage in the peri and post-operative periods and TGN 255 therefore has the potential to provide clinical anticoagulation during CPB without the need for a reversal agent. Echocardiography demonstrated good cardiac function following bypass procedures. The results of this study demonstrate that TGN 255 provides an anticoagulant profile well suited to the coronary bypass setting. By virtue of its predictable anticoagulant efficacy and controlled duration of action, TGN 255 offers potential in on pump and off pump coronary bypass procedures.

### Example 42

### Comparative Stability

The stability of TRI 50c sodium salt and TRI 50c sodium salt calcium salt have been studied in studies of similar design and conditions. In both studies the active pharmaceutical ingredient was stored in grip sealed double bags within a PE/PP screw cap closed cylinder. The packaging allows moisture transfer and the study was designed to allow the investigation into the effects of moisture and oxygen on the stability of these TRI 50c salts.

The results observed after 6 months storage are summarised in the tables below.

Results Sodium Salt, Data in % w/w

| | | | T = 1, | T = 1, | | T = 3, | T = 3, | T = 12, |
|---|---|---|---|---|---|---|---|---|
| | | T = 1, | 25° C./ | 40° C./ | T = 3, | 25° C./ | 40° C./ | 25° C./ |
| | T = 0 | −20° C. | 60% r.h. | 75% r.h. | −20° C. | 60% r.h. | 75% r.h | 60% r.h. |
| Appearance | Powdery | powdery | powdery | powdery | powdery | powdery | viscous | |
| Color | White | white | white | white | white | white | brown | |
| Tri50c (w/w %) | 101.1 | 103.1 | 99.5 | 90.3 | 102.5 | 95.3 | 48.6 | 70.5 |
| Tri50c (w/w %, | 101.5 | 104.3 | 103.6 | 94.5 | — | 100.4 | 52.2 | 71.2 |
| LOD, corr.) | | | | | | | | |

Results Calcium Salt, Data in % w/w

| | | | T = 1, | T = 1, | | T = 3, | T = 3, | |
|---|---|---|---|---|---|---|---|---|
| | | T = 1, | 25° C./ | 40° C./ | T = 3, | 25° C./60% | 40° C./ | T = 12 25° C./ |
| | T = 0 | −20° C. | 60% r.h. | 75% r.h. | −20° C. | r.h. | 75% r.h. | 60% r.h. |
| Appearance | powdery, | powdery | powdery | powdery | powdery | powdery | powdery | |
| | odourless | | | | | | | |
| Color | white | white | white | white | white | white | white | |
| TRI 50c (% peak | 99.4 | 99.1 | 98.3 | 95.2 | 99.2 | 97.5 | 71.2 | 96.9 (94.8 w/w) |
| area) | (99.7) | (102.7)^{x} | (101.5) | (100.6) | (103.0)^{x} | (104.3) | (82.0) | |
| Tri50c (w/w %, | | | | | | | | |
| LOD corrected) | | | | | | | | |

LOD = loss on drying

### Discussion

The data in this example indicate that calcium salts of boronic acids are more stable than the corresponding sodium salts. It is contemplated that the same benefit may be provided by zinc.

It will be appreciated from the foregoing that the disclosure provides boronic acid salts useful for pharmaceutical purposes and which feature one or more of the following attributes: (1) improved hydrolytic stability; (2) improved stability against deboronation; and (3), in any event, not suggested by the prior art.

The selection of active ingredient for a pharmaceutical composition is a complex task, which requires consideration not only of biological properties (including bioavailability) but also of physicochemical properties desirable for processing, formulation and storage. Bioavailability itself is dependent on various factors, often including in vivo stability, solvation properties and absorption properties, each in turn potentially dependent on multiple physical, chemical and/or biological behaviours.

1. A method for preventing unwanted coagulation during a Coronary Artery Bypass Graft (CABG) procedure, comprising administering a therapeutically effective amount of a compound selected from boronic acids of formula (I), and salts, prodrugs and prodrug salts thereof and combinations of the aforegoing: 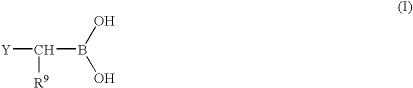
wherein
Y comprises a moiety which, together with the fragment —CH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen, wherein halogen is F, Cl, Br or I.

2. The method of claim 1 wherein R⁹ is an alkoxyalkyl group.

3. The method of claim 1, wherein Y comprises an amino group bonded to structural fragment —CH(R⁹)—B(OH)₂ and a hydrophobic moiety which is linked to said amino group and which, together with said structural fragment, has affinity for the substrate binding site of thrombin.

4. The method of claim 1 wherein the boronic acid is of the formula (II): 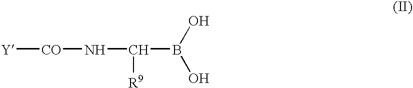
wherein
Y′ comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is, 2, 3, 4 or 5 and W is —OH or halogen, and when Y′CO— is an optionally N-terminally protected dipeptide which binds to the S3 and S2 binding sites of thrombin, the peptide linkages in the acid are optionally and independently N-substituted by a C₁-C₁₃ hydrocarbyl group optionally containing in-chain or in-ring nitrogen, oxygen or sulfur and optionally substituted by a substituent selected from halo, hydroxy and trifluoromethyl.

5. The method of claim 4 wherein Y′CO— comprises an amino acid which binds to the S2 subsite of thrombin, the amino acid being N-terminally linked to a moiety which binds the S3 subsite of thrombin.

6. The method of claim 4 wherein Y′CO— is an optionally N-terminally protected dipeptide which binds to the S3 and S2 binding sites of thrombin.

7. The method of claim 5 wherein Y′CO— is N-terminally protected.

8. The method of claim 6 wherein the S3-binding amino acid residue is of (R)-configuration, the S2-binding residue is of (S)-configuration, and the fragment —NHCH(R⁹)—B(OH)₂ is of (R)-configuration.

9. The method of claim 8 wherein the boronic acid has a Ki for thrombin of about 100 nM or less.

10. The method of claim 1, wherein the boronic acid is of formula (III): 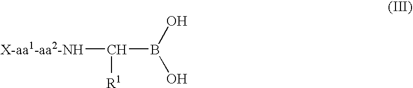
where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members and;
R¹ is a group of the formula —(CH₂)ₛ-Z, where s is 2, 3 or 4 and Z is —OH, —OMe, —OEt or halogen.

11. The method of claim 10 wherein aa¹ is selected from Phe, Dpa and wholly or partially hydrogenated analogues thereof.

12. The method of claim 10 wherein aa² is a residue of an imino acid of formula (IV) 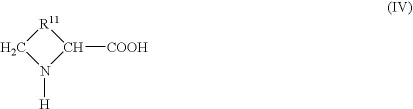
where R¹¹ is —CH₂—, —CH₂—CH₂—, —CH═CH—, —S—CH₂—, —S—C(CH₃)₂— or —CH₂—CH₂—CH₂—, which residue, when the ring contained therein is 5- or 6-membered, is optionally substituted at one or more —CH₂— groups by from 1 to 3 C₁-C₃ alkyl groups.

13. The method of claim 10 wherein aa¹ is of (R)-configuration, aa² is of (S)-configuration the fragment —NH—CH(R¹)—B(OH)₂ is of (R)-configuration.

14. The method of claim 10 wherein R¹ is 2-bromoethyl, 2-chloroethyl, 2-methoxyethyl, 3-bromopropyl, 3-chloropropyl or 3-methoxypropyl.

15. The method of claim 10 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by one or more substituents selected from halogen, amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group.

16. The method of claim 10 wherein the boronic acid is of formula (VIII):
X—(R)-Phe-(S)-Pro-(R)-Mpg-B(OH)₂ (VIII).

17. The method of claim 10 wherein the boronic acid is of the formula:
Cbz-(R)-Phe-(S)-Pro-(R)-boroMpg-OH.

18. The method of claim 1 wherein the prodrugs are boronic acid derivatives capable of hydrolysing to release the free boronic acid.

19. The method of claim 1 wherein the prodrugs comprise a cyclic derivative in which the boronyl group is derivatised to form a moiety of the formula: 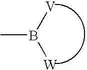
where V and W are heteroatoms and the arcuate line represents a linear or branched chain of atoms.

20. The method of claim 1 wherein the compound does not comprise a choline or ammonium salt.

21. The method of claim 1 wherein the compound is a base addition salt of the boronic acid.

22. The method of claim 21 wherein the compound comprises a salt of the boronic acid with an alkali metal or a strongly basic organic nitrogen-containing compound.

23. The method of claim 21 wherein the compound comprises a salt of the boronic acid with a metal.

24. The method of claim 23 wherein the metal is sodium or lithium.

25. The method of claim 23 wherein the metal is an alkaline earth metal.

26. The method of claim 1 wherein the compound comprises boronate ions derived from the boronic acid and has a stoichiometry consistent with the boronate ions carrying a single negative charge.

27. The method of claim 1 wherein the compound is administered intravenously.

28. The method of claim 1 further comprising administering a cardiovascular treatment agent selected from a lipid-lowering drug, a fibrate, niacin, a statin, a CETP inhibitor, a bile acid sequestrant, an anti-oxidant, a IIb/IIIa antagonist, an aldosterone inhibitor, an A2 antagonist, an A3 agonist, a beta-blocker, acetylsalicylic acid, a loop diuretic, an ace inhibitor, an antithrombotic agent with a different mechanism of action, an antiplatelet agent, a thromboxane receptor and/or synthetase inhibitor, a fibrinogen receptor antagonist, a prostacyclin mimetic, a phosphodiesterase inhibitor, an ADP-receptor (P2 T) antagonist, a thrombolytic, a cardioprotectant and a COX-2 inhibitor.

29. A method for the prevention of thrombosis during coronary artery bypass grafting, comprising administering intravenously to a subject a therapeutically effective amount of a selective thrombin inhibitor which is a boronic acid having a neutral aminoboronic acid residue capable of binding to the thrombin S1 subsite linked through a peptide linkage to a hydrophobic moiety capable of binding to the thrombin S2 and S3 subsites, or is a salt, prodrug or prodrug salt of such an acid.

30. A cardiopulmonary bypass apparatus comprising a cardiopulmonary bypass circuit, wherein the circuit, or a part thereof, is coated with a compound as defined in claim 1.

31. A method or preventing unwanted coagulation or thrombosis during surgery, comprising administering a therapeutically effective amount of a compound selected from boronic acids as defined in claim 1, and acid addition salts and prodrugs thereof.

32. The method of claim 31, wherein the prodrugs are boronic acid derivatives capable of hydrolysing to release the free boronic acid.

33. The method of claim 1 wherein the compound is administered to a patient undergoing such a procedure or into an extracorporeal blood circuit connected to such a patient.

34. A method for preventing unwanted coagulation during a procedure selected from the group consisting of surgery involving an extracorporeal blood circuit and surgery not involving an extracorporeal blood circuit, comprising administering a therapeutically effective amount of a compound selected from the group consisting of boronic acids as defined in claim 1, and acid addition salts and prodrugs thereof to a patient undergoing such a procedure or into an extracorporeal blood circuit connected to such a patient.

35. The method of claim 34 wherein the boronic acid is of formula (I): 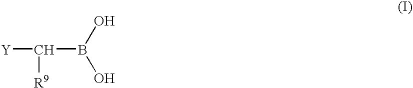
wherein
Y comprises a moiety which, together with the fragment —CH(R⁹)—B(OH)₂, has affinity for the substrate binding site of thrombin and wherein Y comprises an amino acid which binds to the S2 subsite of thrombin, the amino acid being N-terminally linked to a moiety which binds the S3 subsite of thrombin, R⁹ is an alkoxyalkyl group; and wherein the S3-binding amino acid residue is of (R)-configuration, the S2-binding residue is of (S)-configuration, and the fragment —CH(R⁹)—B(OH)₂ is of (R)-configuration.

36. The method of claim 34 wherein the boronic acid is of formula (III): 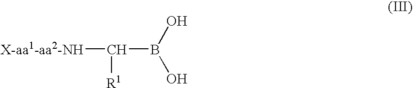
where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members; and
R¹ is a group of the formula —(CH₂)ₛ-Z, where s is 2, 3 or 4 and Z is —OH, —OMe, —OEt or halogen, wherein halogen is F, Cl, Br or I.

37. The method of claim 34 wherein the boronic acid is a compound designated TRI 50c of the following formula:
Cbz-(R)-Phe-(S)-Pro-(R)-Mpg-B(OH)₂.

38. A method of for preventing thrombosis during Coronary Artery Bypass Grafting, comprising administering a therapeutically effective amount of a composition of matter comprising:
a) a first species selected from the group consisting of a boronic acid of formula (I) below, said acid when in the form of a boronate anion thereof, and an equilibrium form of said boronic acid and of said boronate ion, and combinations thereof: 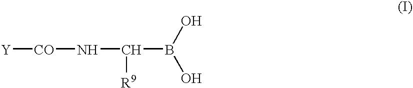
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen, wherein halogen is F, Cl, Br or I; and
(b) a second species selected from the group consisting of pharmaceutically acceptable metal ions and strongly basic organic nitrogen-containing compounds.

39. The method of claim 11 wherein aa¹ is selected from Dpa, Phe, Dcha and Cha.

40. The method of claim 12 wherein aa² is an (S)-proline residue.

41. The method of claim 15 wherein the 5 to 13-membered cyclic group is aromatic or heteroaromatic.

42. The method of claim 22 wherein the strongly basic organic nitrogen-containing compound is guanidine, a guanidine analog or an amine.
